(19) United States
(12) Patent Application Publication
(10) Pub. No.: US 2025/0295579 A1
(43) Pub. Date: Sep. 25, 2025

(54) SKIN CARE COMPOSITION AND USES THEREOF

(71) Applicant: S-BIOMEDIC NV, BEERSE (BE)

(72) Inventors: Laura Gomez Martin-Ambrosio, Leuven (BE); Miquel Rozas Belmonte, Antwerp (BE); Amine Zorgani, Brussels (BE); Bernhard Paetzold, Berchem (BE); Willy Verheyen, Vosselaar (BE)

(73) Assignee: S-BIOMEDIC NV, BEERSE (BE)

(21) Appl. No.: 18/728,031

(22) PCT Filed: Jan. 12, 2023
(86) PCT No.: PCT/EP2023/050583

§ 371 Date: Jul. 10, 2024

(30) Foreign Application Priority Data
Jan. 12, 2022 (EP) 22151098.5

(51) Int. Cl. A61K 8/99; A61K 8/37; A61K 8/39; A61K 35/74; A61Q 5/00; A61Q 19/00; A61Q 19/08

(52) U.S. Cl. CPC A61K 8/99; A61K 8/375; A61K 8/39; A61K 35/74; A61Q 5/006; A61Q 19/00; A61Q 19/008; A61Q 19/08; A61K 2800/84

(57) ABSTRACT

The present invention generally relates to the field of skin care. More particularly, the invention relates to a cosmetic or therapeutic skin care composition comprising live bacteria of at least one Cutibacterium acnes (C. acnes) strain in combination an ester of a polyethylene glycol and a fatty acid. Also a kit of parts configured to prepare such skin care composition is provided. The invention also provides the skin care composition or the kit of parts for use in the treatment and/or prevention of skin conditions or skin diseases. Also methods for improving the appearance of the skin, for modulating the sebum production of skin cells or for maintaining a healthy or youthly appearance of the skin are envisaged.

### RELATED APPLICATIONS

This application is a national stage filing under 35 U.S.C. § 371 of International Patent Application Serial Number PCT/EP2023/050583, filed Jan. 12, 2023, entitled “SKIN CARE COMPOSITION AND USES THEREOF,” which claims priority to European Application Serial Number 22151098.5, filed Jan. 12, 2022, the entire contents of each of which is herein incorporated by reference in its entirety.

### REFERENCE TO AN ELECTRONIC SEQUENCE LISTING

The contents of the electronic sequence listing (S190470003US00-SEQ-OMJ.xml; Size: 11,531 bytes; and Date of Creation: Jul. 8, 2024) is herein incorporated by reference in its entirety.

### FIELD OF THE INVENTION

The present invention generally relates to the field of skin care. More particularly, the invention relates to cosmetic or therapeutic skin care compositions and kits of parts comprising live bacteria of at least one Cutibacterium acnes (C. acnes). The products of the invention can be used in the treatment and/or prevention of skin conditions or skin diseases, as well as in cosmetic methods.

### BACKGROUND OF THE INVENTION

The human body is host to a highly complex and rich microbial community. These microorganisms are generally harmless and contribute to a healthy state by producing vitamins, cooperating with digesting food, or stimulating the immune system. The human microbiota mainly resides on the surface and in deep layers of skin, in the saliva and oral mucosa, in the conjunctiva, and in the gastrointestinal tracts.

The skin microbiota plays a major role in the barrier function of the skin and consequently also in human skin health and disease. The skin is colonized by a large number of microorganisms, most of them being beneficial or harmless. However, the skin microbiome has specific compositions in disease states of the skin that are different to healthy skin. Diseases such as acne vulgaris are associated with strong alterations of the microbiome. It has further been shown that major alterations occur in the skin microbiota during ageing of the skin. Although the skin microbial composition of healthy subjects has been found to remain largely stable over time during adulthood, age-related physiologic changes—particular alterations in sebum secretion and immune function and a decrease in sweat—may affect the skin microbiome of older individuals.

Alterations in the skin microbiota have thus been observed in both skin diseases, such as acne, as well as in the ageing skin. A key role herein seems to be for the anaerobic gram-positive bacterium Cutibacterium acnes (C. acnes; formerly known as Propionibacterium acnes). C. acnes bacteria decompose the sebum to glycerine and fatty acids, thereby further inducing the production of sebum in the sebaceous glands and destroying the follicle walls in the skin. This results in inflammation of the skin and formation of pimples, pustules, nodules and cysts which often heal only with scarring.

On the other hand, C. acnes is one of the most abundant species of micro-organisms on the skin, which suggests that it has co-evolved with humans and therefore, its presence may confer skin benefits. This hypothesis is strengthened by the exclusive niche that C. acnes inhabits—it is nearly the sole inhabitant of the sebaceous hair follicle. It has therefore been suggested that modulation of the skin microbiome in order to restore the microbiome into a healthy microbiome can be achieved by administering C. acnes bacteria to the skin. For example, WO 2016/172196 discloses a method of treating acne in a subject by administering a composition comprising one or more live C. acnes strains to the skin of the subject. Similarly, WO 2018/073651 discloses a composition for acne treatment comprising two or more different C. acnes strains, including C. acnes strain C3 and/or K8.

### SUMMARY

The present invention is at least in part based on the inventors' discovery that an ester of a polyethylene glycol and a fatty acid can stimulate and boost the growth of at least one C. acnes strain. C. acnes is one of the most abundant microorganisms in the skin microbiome and its presence contributes to a healthy skin. Further, stimulation of its growth has been shown to restore and maintain a healthy balance in the skin microbiome, and may also be used to prevent or treat certain skin diseases, such as acne, oily skin or eczema. In the present invention, it is now found that the growth of C. acnes can further be stimulated by the presence of an ester of a polyethylene glycol (PEG) and a fatty acid. As such, the combination of at least one C. acnes strain and an ester of a polyethylene glycol and a fatty acid in a skin care composition for topical administration or in a kit of parts can be used to improve the appearance of the skin, to maintain a healthy appearance of the skin, or to treat and/or prevent a skin disease or skin condition.

In an aspect of the invention, a skin care composition for topical administration is provided. The skin care composition comprises live bacteria of at least one C. acnes strain and a polyethylene glycol ester of a fatty acid.

In another aspect, the invention provides a kit of parts configured to prepare said skin care composition, and wherein the kit of parts comprises at least one C. acnes strain and a composition comprising a polyethylene glycol ester of a fatty acid.

Another aspect of the invention provides the skin care composition or the kit of parts as disclosed herein for use in the treatment and/or prevention of a condition or disease selected from the group consisting of acne, oily skin, progressive macular hypomelanosis, dandruff, atopic eczema, atopic dermatitis and rosacea. In a particular embodiment, the skin care composition or the kit of parts as disclosed herein are for use in the treatment and/or prevention of acne or oily skin; preferably acne.

Another aspect of the invention provides the skin care composition or the kit of parts as disclosed here for use in the treatment and/or prevention of an oxidative stress-associated skin disease.

Another aspect provides a cosmetic method for improving the appearance of the skin of a subject wherein the method comprises topical administration of the skin care composition or of the kit of parts as disclosed herein to an area of the subject's skin.

A related aspect provides a cosmetic method for modulating the sebum production of skin cells of a subject wherein the method comprises topical administration of the skin care composition or of the kit of parts as disclosed herein to an area of the subject's skin.

A further aspect provides a cosmetic method for maintaining a healthy or youthful appearance of the skin in a subject, wherein the method comprises topical administration of the skin care composition or of the kit of parts as disclosed herein to an area of the subject's skin.

Another aspect provides a method for stimulating or boosting the growth of at least one endogenous bacterial strain on the skin of a subject, wherein the method comprises topical administration of the skin care composition or kit of parts as disclosed herein.

Another aspect provides a method for stimulating or boosting the growth of at least one endogenous C. acnes bacterial strain on the skin of a subject, wherein the method comprises topical administration of a skin care composition comprising an ester of a polyethylene glycol and a fatty acid to the skin of the subject.

Another aspect provides a method for stimulating or boosting the growth of at least one C. acnes bacterial strain in vitro, wherein the method comprises administration of a composition comprising a polyethylene glycol ester of a fatty acid to said at least one C. acnes bacterial strain in vitro.

The above and further aspects and preferred embodiments of the invention are described in the following sections and in the appended claims. The subject-matter of appended claims is hereby specifically incorporated in this specification.

### BRIEF DESCRIPTION OF DRAWINGS

The following description of the figures of specific embodiments of the invention is merely exemplary in nature and is not intended to limit the present teachings, their application or uses.

FIG. 1. Effect of glycerol on the growth of C. acnes strains. The data are represented as the percentage of baseline change in optical density (OD₆₀₀) from peptone control.

FIG. 2. Effect of sorbitol on the growth of C. acnes strains. The data are represented as the percentage of baseline change in OD₆₀₀ from peptone control.

FIG. 3. Effect of sodium lactate on the growth of C. acnes strains. The data are represented as the percentage of baseline change in OD₆₀₀ from peptone control.

FIG. 4. Effect of PEG40 stearate on the growth of C. acnes strains. The data are represented as the percentage of baseline change in OD₆₀₀ from peptone control.

FIG. 5. Effect of sodium cetaryl sulphate on the growth of C. acnes strains. The data are represented as the percentage of baseline change in OD₆₀₀ from peptone control.

FIG. 6. Effect of different concentrations of different PEG components on the growth of the C. acnes A1 strain.

FIG. 7. Effect of different concentrations of different PEG components on the growth of the C. acnes D1 strain.

FIG. 8. Effect of different concentrations of different PEG components on the growth of the C. acnes H1 strain.

FIG. 9. Effect of different concentrations of different PEG components on the growth of the C. acnes K8 strain.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The present invention addresses a need for novel skin care products which have been formulated in a way to be compatible with the application of bacteria and which do not inhibit, and even stimulate, the outgrowth of these bacteria after application of the product to the skin.

With the present invention, the inventors surprisingly found that the growth of one or more C. acnes bacteria is stimulated and boosted in the presence of one or more compounds selected from the group consisting of a polyethylene glycol ester of a fatty acid, glycerol, sorbitol, lactic acid or a salt thereof, and cetearyl sulphate. These one or more compounds can be added to the formulation of the final skin care product, or they can be applied as a separate skin care formulation before application of the bacteria to the skin. As such, the combination of at least one C. acnes strain and one or more of these compounds in a skin care composition for topical administration or in a kit of parts can be used to improve the appearance of the skin, to maintain a healthy appearance of the skin, or to treat and/or prevent a skin disease or skin condition. Additionally, the one or more compounds can also be added to at least one C. acnes strain in vitro to stimulate the bacterial growth in vitro.

Particularly advantageous is the combination of at least one C. acnes strain and a polyethylene glycol ester of a fatty acid. The combination of at least one C. acnes strain and a polyethylene glycol ester of a fatty acid can thus be applied in a skin care composition for topical administration or in a kit of parts to improve the appearance of the skin, to maintain a healthy appearance of the skin, or to treat and/or prevent a skin disease or skin condition.

Thus, in a first aspect the present invention relates to a skin care composition for topical administration to the skin, said composition comprising, consisting essentially of, or consisting of live bacteria of at least one C. acnes strain and a polyethylene glycol ester of a fatty acid.

Also provided is a skin care composition for topical administration to the skin, said composition comprising, consisting essentially of, or consisting of live bacteria of at least one C. acnes strain and a polyethylene glycol ester of a fatty acid or one or more compounds selected from the group consisting of glycerol, sorbitol, lactic acid or a salt thereof, and cetearyl sulphate. In certain embodiments, the composition may comprise any mixture of two or more of said compounds.

In another aspect, the present invention relates to a kit of parts configured to prepare a skin care composition as disclosed herein, wherein the kit of parts comprises live bacteria of at least one C. acnes strain and a composition comprising an ester of a polyethylene glycol and a fatty acid.

Further provided is a kit of parts configured to prepare a skin care composition as disclosed herein, wherein the kit of parts comprises live bacteria of at least one C. acnes strain and a composition comprising a polyethylene glycol ester of a fatty acid or one or more compounds selected from the group consisting of glycerol, sorbitol, lactic acid or a salt thereof, and cetearyl sulphate. In certain embodiments, the composition may comprise any mixture of two or more of said compounds.

In certain embodiments, the bacteria in the skin care composition or kit of parts may be lyophilized or spray-dried live bacteria. This means that viable bacteria have been subjected to a drying process that maintains their viability, but reduces their metabolic processes to a minimum. In lyophilized or spray-dried form, the bacteria can be stored for months or even years. Once they are applied to the skin, such as the human skin, the metabolism of the bacteria is reactivated such that they resume growth. They propagate on the skin surface and displace pathogenic bacterial strains, thereby recovering a diverse, healthy and balanced skin microbiome.

In one embodiment, the live C. acnes bacteria are present in spray-dried form. The principle of spray drying is based on the dispersion of a solution into fine droplets which are introduced into a flow of hot air. The solvent evaporates from the substrate droplets so that dry product clusters remain. Standard spray drying devices can be used, such as the Mini Spray Dryer B-290 from Büchi Labortechnik GmbH (Essen, Germany) or the Mobile Minor™ Spray Dryer from GEA (Berlin, Germany).

In one embodiment, the live C. acnes bacteria are present in freeze-dried or lyophilized form. Freeze drying or lyophilization is a process which includes freezing the product, reducing the pressure and adding heat to allow the frozen water in the material to sublimate. Various methods can be applied for freezing the product. For example, freezing can be achieved by using a standard freezer or a chilled bath. Cooling the product below its triple point ensures that sublimation will occur upon heating. To prevent the formation of large crystals that may damage the structure of the product to be dried, freezing is done rapidly. About 95% of the water in the product is removed when the frozen water sublimates. Most materials can be dried to 1-5% w/w residual moisture. Standard freeze drying devices can be used, such as the Lyovac™ devices from GEA (Berlin, Germany), the Gamma 2-20 Freeze dryer LCM-1 from Christ (Osterode am Harz, Germany), or the Christ Martin™ Alpha 1-2 Lyophilisator from Fisher Scientific GmbH (Schwerte, Germany).

In some embodiments, the skin care composition or the kit of parts comprises at least one C. acnes strain, preferably at least one C. acnes strain selected from the group consisting of single locus sequence typing (SLST) type strains A1, D1, A5, C1, C3, H1, H2, H3, K1, K2, K4, K6, K8, K9, L1, and F4. In some embodiments, the skin care composition or kit of parts comprises lyophilized or spray dried live bacteria of at least one C. acnes strain selected from SLST type A1, D1, H1, and K8. In some embodiments, the skin care composition or kit of parts comprises lyophilized or spray-dried live bacteria of at least one C. acnes SLST type A1 strain. In some embodiments, the skin care composition or kit of parts comprises lyophilized or spray-dried live bacteria of at least one C. acnes SLST type D1 strain. In some embodiments, the skin care composition or kit of parts comprises lyophilized or spray-dried live bacteria of at least one C. acnes SLST type H1 strain. In some embodiments, the skin care composition or kit of parts comprises lyophilized or spray-dried live bacteria of at least one C. acnes SLST type K8 strain.

In some embodiments, the concentration of each C. acnes strain, such as each lyophilized or spray-dried C. acnes strain, is at least 0.5% w/v of the skin care composition. In some embodiments, when more than one C. acnes strain is present in the skin care composition or kit of parts, each strain is at approximately equal concentrations within the composition. In some embodiments, one C. acnes strain is present at a higher concentration than the other one or more C. acne strains within the composition.

It has particularly been shown by the inventors that an ester of a polyethylene glycol and a fatty acid can stimulate and boost the growth of the live bacteria, such as each lyophilized or spray-dried C. acnes strain, and that such ester of a polyethylene glycol and a fatty acid is present in the skin care composition or kit of parts as disclosed herein.

Further, the one or more compounds that stimulate and boost the growth of the live bacteria, such as each lyophilized or spray-dried C. acnes strain, and that are present in the skin care composition or kit of parts as disclosed herein can be selected from the group consisting of a polyethylene glycol ester of a fatty acid, glycerol, sorbitol, lactic acid or a salt thereof, and cetearyl sulphate. In some embodiments, a combination of two or more of these compounds can be used in the skin care composition or kit of parts as disclosed herein, for example a combination of two, three, four or all of the compounds selected from the group consisting of a polyethylene glycol ester of a fatty acid, glycerol, sorbitol, lactic acid or a salt thereof, and cetearyl sulphate.

In some embodiments, the skin care composition or kit of parts thus comprises, consists essentially of, or consists of live bacteria of at least one C. acnes strain, such as lyophilized or spray-dried live bacteria of at least one C. acnes strain, and a polyethylene glycol ester of a fatty acid. The fatty acid can be an unsaturated or a saturated fatty acid. In some further embodiments, the skin care composition or kit of parts comprises a polyethylene glycol ester of a saturated fatty acid. In some other embodiments, the skin care composition or kit of parts comprises a polyethylene glycol ester of a C₈-C₂₄ fatty acid, preferably a C₁₆-C₁₈ fatty acid, such as a saturated or unsaturated C₁₆-C₁₈ fatty acid. In some further embodiments, the skin care composition or kit of parts comprises a polyethylene glycol ester of a saturated C₁₆-C₁₈ fatty acid.

The polyethylene glycol ester of a fatty acid can be obtained in particular from an acid comprising a saturated or unsaturated linear alkyl chain containing 8 to 24, and preferably 16 to 18 carbon atoms. These ranges for the number of carbon atoms include all specific values and subranges therebetween, such as 10, 12, 14, 16, 18, and 20 carbon atoms, preferably 16 or 18 carbon atoms. The fatty acid is thus preferably a C₁₆-C₁₈ fatty acid, such as palmitic acid, oleic acid, or stearic acid. In most preferred embodiment, the fatty acid is stearic acid. Stearic acid, also referred to as octadecanoic acid or stearate, is a saturated long chain fatty acid with an 18-carbon backbone. Oleic acid, also referred to as cis-9-octadecanoic acid or oleate, is an octadic-9-enoic acid in which the double bond at C-9 has Z (cis) stereochemistry. Palmitic acid, also referred to as hexadecanoic acid, cetylic acid or palmitate, is a straight-chain, 16 carbon, saturated long-chain fatty acid.

The polyethylene glycol ester of a fatty acid as envisaged herein is thus a polyethylene glycol polymer (also known as polyethylene oxide or polyoxyethylene, and encompassing oligomers or polymers of ethylene oxide) with a fatty acid attached via an ester linkage to some or all terminal hydroxyl end groups of the polyethylene glycol polymer molecules (such as with respect to an individual PEG linear chain, to one or both terminal hydroxyl end groups). Contemplated for use herein are PEGs composed of linear ethylene oxide oligomer or polymer chains, as well as PEGs with branched, Y-shaped, or multi-arm geometries. The polyethylene glycols of the polyethylene glycol ester can have a wide variety of average molecular masses, such as for example average molecular mass in the range 190-210 Da (PEG 200), about 285-315 Da (PEG 300), about 380-420 Da (PEG 400), about 570-630 Da (PEG 600), about 855-900 Da (PEG 900), about 950-1050 Da (PEG 1000), about 1900-2200 Da (PEG 2000), about 2700-3300 Da (PEG 3000), about 3500-4500 Da (PEG 4000), or about 7000-9000 Da (PEG 8000).

In some embodiments, the polyethylene glycol ester of a fatty acid is a polyethylene glycol ester of a fatty acid wherein the fatty acid is a C₈-C₂₄ fatty acid, preferably a C₁₆-C₁₈ fatty acid. Even more preferably, said fatty acid is palmitic acid, oleic acid or stearic acid. In some further preferred embodiments, the polyethylene glycol ester of a fatty acid is a polyethylene glycol ester of a stearic acid.

Particularly preferably, the polyethylene glycol ester of a fatty acid is a PEG-40 stearate, also known as polyoxyethylene (40) stearate or polyoxyl 40 stearate. This designation is commonplace in the art, and the substance can also be referred to by its CAS number 9004-99-3 or IUPAC name: Poly(oxy-1,2-ethanediyl), .alpha.-(1-oxooctadecyl)-.omega.-hydroxy- (40 mol EO average molar ratio)).

As further exemplified in the experimental sections, the inventors found that the presence of a polyethylene glycol ester of a fatty acid, such as a PEG-40 stearate, stimulated and boosted the growth of at least one C. acnes strain. More specifically, the presence of a polyethylene glycol ester of a fatty acid, such as a PEG-40 stearate, stimulated and boosted the growth of the C. acnes SLST type A1 strain, the C. acnes SLST type D1 strain, the C. acnes SLST type H1 and K8 strains. Therefore, in some embodiments, the skin care composition or kit of parts as disclosed herein comprises (optionally lyophilized or spray-dried) live bacteria of at least one C. acnes strain selected from the C. acnes SLST type A1 strain, the C. acnes SLST type D1 strain, the C. acnes SLST type H1 strain or the C. acnes SLST type K8 strain, and a polyethylene glycol ester of a fatty acid as disclosed herein, such as preferably PEG-40 stearate.

In some preferred embodiments, the skin care composition or kit of parts as disclosed herein comprises, consists essentially of, or consists of (optionally lyophilized or spray-dried) live bacteria of the C. acnes SLST type A1 strain, the C. acnes SLST type D1 strain, the C. acnes SLST type H1 strain, the C. acnes SLST type K8 strain, or a mixture thereof, and a polyethylene glycol ester of a fatty acid, such as described above. Particularly preferably, the polyethylene glycol ester of a fatty acid is a PEG-40 stearate. In a particularly preferred embodiment, the skin care composition or kit of parts as disclosed herein comprises, consists essentially of, or consists of (optionally lyophilized or spray-dried) live bacteria of the C. acnes SLST type A1 strain, the C. acnes SLST type D1 strain, the C. acnes SLST type H1 strain, the C. acnes SLST type K8 strain, or a mixture thereof, and a PEG stearate, preferably a PEG-40 stearate.

In some embodiments, the skin care composition or the composition comprised by the kit of parts as disclosed herein comprises in between 0.01 wt % and 10 wt %, preferably in between 0.05 wt % and 5 wt %, preferably in between 0.06 wt % and 5 wt % of the polyethylene glycol ester of a fatty acid, preferably of a PEG stearate, even more preferably of a PEG-40 stearate, relative to the total weight of the skin care composition. For example, the skin care composition thus comprises 0.05 wt %, 0.06 wt %, 0.07 wt %, 0.08 wt %, 0.09 wt %, 0.10 wt %, 0.20 wt %, 0.30 wt %, 0.40 wt %, 0.50 wt %, 0.60 wt %, 0.70 wt %, 0.80 wt %, 0.90 wt %, 1 wt %, 2 wt %, 3 wt %, 4 wt % or 5 wt % of the polyethylene glycol ester of a fatty acid relative to the total weight of the skin care composition or of the composition comprised by the kit of parts.

Also provided herein is a skin care composition or kit of parts that comprises, consists essentially of, or consists of live bacteria of at least one C. acnes strain, such as lyophilized or spray-dried live bacteria of at least one C. acnes strain, and glycerol. Glycerol, also referred to as glycerine, is a colourless, odourless and viscous polyol compound. Glycerol is frequently used because of its humectant properties. As shown in the experimental section, the inventors now found that glycerol surprisingly also boosts and stimulates the growth of at least one C. acnes strain.

Further provided is a skin care composition for topical administration to the skin, wherein the skin care composition comprises (optionally lyophilized or spray-dried) live bacteria of at least one C. acnes strain, and glycerol. In some other embodiments, a kit of parts is provided to prepare a skin care composition wherein the kit of parts comprises (optionally lyophilized or spray-dried) live bacteria of at least one C. acnes strain and a composition comprising glycerol. Preferably the skin care composition or the composition comprised by the kit of parts comprises in between about 0.01 wt % to 10 wt % glycerol, more preferably in between 0.1 wt % and 5 wt % glycerol, even more preferably in between 0.5 wt % and 2 wt % glycerol, such as for example about 0.50 wt %, 0.60 wt %, 0.70 wt %, 0.80 wt %, 0.90 wt %, 1.0 wt %, 1.5 wt %, or 2.0 wt %, of glycerol, relative to the total weight (w/w) of the skin care composition or of the composition comprised by the kit of parts.

In some embodiments, the skin care composition or kit of parts comprises at least one C. acnes strain, preferably at least one C. acnes strain selected from the group consisting of single locus sequence typing (SLST) type strains A1, D1, A5, C1, C3, H1, H2, H3, K1, K2, K4, K6, K8, K9, L1, and F4, and glycerol. In some embodiments, the skin care composition or kit of parts comprises (optionally lyophilized or spray dried) live bacteria of at least one C. acnes strain selected from SLST type A1, D1, H1, and K8, and glycerol. In some further embodiments, the skin care composition or kit of parts comprises (optionally lyophilized or spray dried) live bacteria of the C. acnes SLST type K8 strain and glycerol. In some other embodiments, the skin care composition or kit of parts comprises (optionally lyophilized or spray dried) live bacteria of the C. acnes SLST type D1 strain or of the C. acnes SLST type H1 strain, or a mixture thereof, and glycerol.

In some embodiments, the skin care composition or kit of parts thus comprises, consists essentially of, or consists of live bacteria of at least one C. acnes strain, such as lyophilized or spray-dried live bacteria of at least one C. acnes strain, and sorbitol. Sorbitol, also commonly known as glucitol, is a low molecular weight sugar alcohol. Sorbitol is frequently used because of its humectant properties. As shown in the experimental section, the inventors now found that sorbitol surprisingly also boosts and stimulates the growth of at least one C. acnes strain.

In some embodiments, a skin care composition is thus provided for topical administration to the skin, wherein the skin care composition comprises (optionally lyophilized or spray-dried) live bacteria of at least one C. acnes strain, and sorbitol. In some other embodiments, a kit of parts is provided to prepare a skin care composition wherein the kit of parts comprises (optionally lyophilized or spray-dried) live bacteria of at least one C. acnes strain and a composition comprising sorbitol. Preferably the skin care composition or the composition comprised by the kit of parts comprises in between about 0.01 wt % to 10 wt % sorbitol, more preferably in between 0.1 wt % and 10 wt % sorbitol, even more preferably in between 0.50 wt % and 5 wt % sorbitol, such as for example about 0.50 wt %, 0.75 wt %, 1.0 wt %, 1.5 wt %, 2.0 wt %, 2.5 wt %, 3.0 wt %, 4.0 wt % or 5.0 wt % of sorbitol, relative to the total weight (w/w) of the skin care composition or of the composition comprised by the kit of parts.

In some embodiments, the skin care composition or kit of parts comprises at least one C. acnes strain, preferably at least one C. acnes strain selected from the group consisting of single locus sequence typing (SLST) type strains A1, D1, A5, C1, C3, H1, H2, H3, K1, K2, K4, K6, K8, K9, L1, and F4, and sorbitol. In some embodiments, the skin care composition or kit of parts comprises lyophilized or spray dried live bacteria of at least one C. acnes strain selected from SLST type A1, D1, H1, and K8, and sorbitol. In some further preferred embodiments, the skin care composition or kit of parts comprises lyophilized or spray dried live bacteria of the C. acnes SLST type H1 strain and sorbitol.

In some embodiments, the skin care composition or kit of parts comprises, consists essentially of, or consists of live bacteria of at least one C. acnes strain, such as lyophilized or spray-dried live bacteria of at least one C. acnes strain, and lactic acid or a salt thereof. In some, the skin care composition or kit of parts thus comprises, consists essentially of, or consists of (optionally lyophilized or spray-dried) live bacteria of at least one C. acnes strain and lactic acid salt. Preferably, the lactic acid salt is an alkali or earth metal salt of lactic acid and can be preferably selected from the group consisting of sodium lactate, potassium lactate, calcium lactate, and mixtures thereof. In some preferred embodiments, the lactic acid salt is the sodium salt of lactic acid, i.e., sodium lactate.

In some embodiments, a skin care composition is thus provided for topical administration to the skin, wherein the skin care composition comprises (optionally lyophilized or spray-dried) live bacteria of at least one C. acnes strain, and lactic acid or a salt thereof, preferably a lactic acid salt such as sodium lactate. In some other embodiments, a kit of parts is provided to prepare a skin care composition wherein the kit of parts comprises (optionally lyophilized or spray-dried) live bacteria of at least one C. acnes strain and a composition comprising lactic acid or a salt thereof, preferably a lactic acid salt such as sodium lactate. Preferably the skin care composition or the composition comprised by the kit of parts comprises in between about 0.01 wt % to 10 wt % lactic acid or salt thereof, more preferably in between 0.1 wt % and 10 wt % lactic acid or salt thereof, even more preferably in between 0.2 wt % and 5 wt % lactic acid or salt thereof, such as for example about 0.20 wt %, 0.30 wt %, 0.40 wt %, 0.50 wt %, 0.75 wt %, 1.0 wt %, 1.5 wt %, 2.0 wt %, 2.5 wt %, 3.0 wt %, 4.0 wt % or 5.0 wt % of lactic acid or salt thereof, relative to the total weight (w/w) of the skin care composition or of the composition comprised by the kit of parts.

In some embodiments, the skin care composition or kit of parts comprises at least one C. acnes strain, preferably at least one C. acnes strain selected from the group consisting of single locus sequence typing (SLST) type strains A1, D1, A5, C1, C3, H1, H2, H3, K1, K2, K4, K6, K8, K9, L1, and F4, and a lactic acid or salt thereof, preferably a lactic acid salt, such as sodium lactate. In some embodiments, the skin care composition or kit of parts comprises (optionally lyophilized or spray dried) live bacteria of at least one C. acnes strain selected from SLST type A1, D1, H1, and K8, and a lactic acid or salt thereof, preferably a lactic acid salt, such as sodium lactate. In some further embodiments, the skin care composition or kit of parts comprises (optionally lyophilized or spray dried) live bacteria of the C. acnes SLST type K8 strain and a lactic acid or salt thereof, preferably a lactic acid salt, such as sodium lactate.

In some embodiments, the skin care composition or kit of parts comprises, consists essentially of, or consists of live bacteria of at least one C. acnes strain, such as lyophilized or spray-dried live bacteria of at least one C. acnes strain, and a cetearyl sulphate. In some embodiments, the skin care composition or kit of parts thus comprises, consists essentially of, or consists of (optionally lyophilized or spray-dried) live bacteria of at least one C. acnes strain and a cetearyl sulphate. Cetearyl compounds, such as cetaryl sulphate or cetearyl phosphate, are often used as emulsifiers. The inventors now found that cetearyl sulphate, in particular sodium cetearyl sulphate, surprisingly boosts and stimulates the growth of at least one C. acnes strain.

In some embodiments, a skin care composition is thus provided for topical administration to the skin, wherein the skin care composition comprises (optionally lyophilized or spray-dried) live bacteria of at least one C. acnes strain, and cetearyl sulphate, preferably sodium cetearyl sulphate. In some other embodiments, a kit of parts is provided to prepare a skin care composition wherein the kit of parts comprises (optionally lyophilized or spray-dried) live bacteria of at least one C. acnes strain and a composition comprising cetearyl sulphate, preferably sodium cetearyl sulphate. Preferably the skin care composition or the composition comprised by the kit of parts comprises in between about 0.001 wt % to 2 wt % cetearyl sulphate, more preferably in between 0.010 wt % and 1.0 wt % cetearyl sulphate, even more preferably in between 0.050 wt % and 0.50 wt % cetearyl sulphate, or between 0.075 wt % and 0.75 wt % cetearyl sulphate such as for example about 0.010 wt %, 0.020 wt %, 0.030 wt %, 0.040 wt %, 0.060 wt %, 0.070 wt %, 0.075 wt %, 0.080 wt %, 0.09 wt %, 0.10 wt %, 0.20 wt %, 0.30 wt %, 0.40 wt %, 0.50 wt %, 0.60 wt %, 0.70 wt % of cetearyl sulphate, relative to the total weight (w/w) of the skin care composition or of the composition comprised by the kit of parts.

In some embodiments, the skin care composition or kit of parts comprises at least one C. acnes strain, preferably at least one C. acnes strain selected from the group consisting of single locus sequence typing (SLST) type strains A1, D1, A5, C1, C3, H1, H2, H3, K1, K2, K4, K6, K8, K9, L1, and F4, and a cetearyl sulphate, preferably sodium cetearyl sulphate. In some embodiments, the skin care composition or kit of parts comprises (optionally lyophilized or spray dried) live bacteria of at least one C. acnes strain selected from SLST type A1, D1, H1, and K8, and a cetearyl sulphate, preferably sodium cetearyl sulphate. In some other embodiments, the skin care composition or kit of parts comprises (optionally lyophilized or spray dried) live bacteria of the C. acnes SLST type H1 strain and a cetearyl sulphate, preferably sodium cetearyl sulphate. In some other embodiments, the skin care composition or kit of parts comprises (optionally lyophilized or spray dried) live bacteria of the C. acnes SLST type D1 strain and a cetearyl sulphate, preferably sodium cetearyl sulphate. In still some other embodiments, the skin care composition or kit of parts comprises (optionally lyophilized or spray dried) live bacteria of the C. acnes SLST type D1 strain and of the SLST type H1, and a cetearyl sulphate, preferably sodium cetearyl sulphate.

The skin care composition or kit of parts of the present invention comprises (optionally lyophilized or spray-dried) live bacteria of at least one strain of the species C. acnes. In some preferred embodiments, the skin care composition or kit of parts may comprise two or more strains of the species C. acnes. For example, the skin care composition may comprise 2, 3, 4, 5, 6, 7, 8, 9, 10, 11, 12, 13, 14, 15, 16, 17, 18, 19, 20 or more than 20 strains of C. acnes. In some embodiments the skin care composition or kit of parts comprises 2, 3, 4 or 5 different strains of C. acnes.

The bacterial species Cutibacterium acnes (C. acnes) was formerly known as Propionibacterium acnes (P. acnes). Based on the results from biochemical and genomic studies, the species was taxonomically reclassified in 2016. C. acnes is a Gram-positive, anaerobic, rod-shaped bacterium which is known to be involved in the development of acne and other pathological conditions. C. acnes is largely commensal and is an abundant colonizer of human skin thriving in the nutritious environment of sebum rich skin. The bacterium causes no symptoms in the majority of carriers, but certain strains have a suggested pro-inflammatory role in acne vulgaris. Moreover, due to its biofilm-forming capacity, the bacterium is frequently isolated from various orthopaedic implant-associated infections.

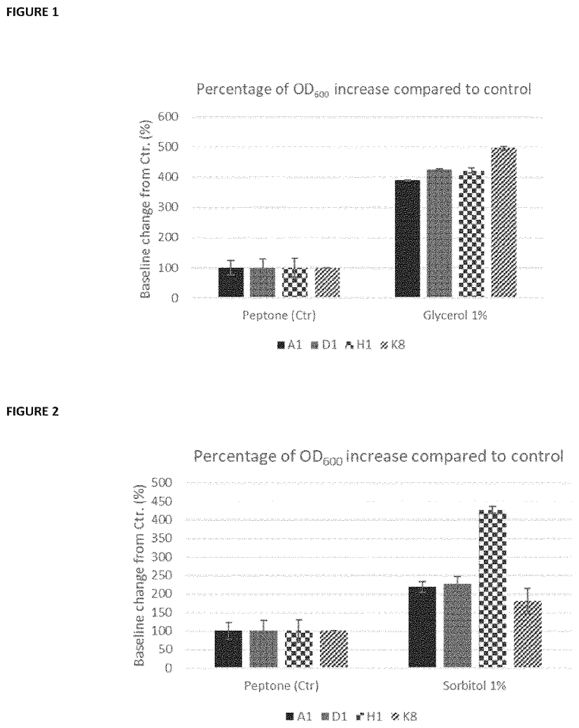

Studies by Johnson and Cummins (1972) first revealed two distinct phenotypes of Propionibacterium acnes, known as types I and II, that could be distinguished based on serological agglutination tests and cell wall sugar analysis. Subsequent studies, involving among others sequence analysis of the recA gene demonstrated that P. acnes comprises four highly distinct evolutionary lineages, known as type or clade IA, IB, II and II that display differences in inflammatory properties, production of virulence determinants and association with various conditions (McDowell at al. 2005; McDowell at al. 2008; Valanne et al. 2005; Lodes et al. 2006). Type IA has been further subdivided into subtypes IA1 and IA2, and type IC has been identified using a multilocus sequence typing scheme (eMLST) based on six housekeeping genes and two putative virulence genes (McDowell et al. 2012). Scholz et al. (2014) later developed a single-locus sequence typing (SLST) scheme for P. acnes strains, involving PCR amplification and DNA sequencing of the target locus PPA2385. A publicly available P. acnes database and SLST allocation tool associated with the SLST scheme described by Scholz et al. is available online at medbac.dk/slst/pacnes. Exemplary SLST types for P. acnes strains include SLST types A1 to A24, B1, C1 to C4, D1 to D3, E1 to E9, F1 to F10, G1, H1 to H5, K1 to K14, and L1 to L6. Other P. acnes strain typing schemes are known as MLST9, MLST8, and Ribotype schemes, reviewed in Scholz et al., especially in FIG. 1. Where SLST types are referred to in this specification, the types according to the SLST typing scheme of Scholz et al., are meant. As further guidance but without limitation, Table 1 on p. 20-31 of WO 2018/073651, incorporated by reference herein, lists several allelic sequences of the target locus PPA2385 used in Scholz et al.'s SLST to identify SLST types of P. acnes strains.

Illustrative but non-limiting strains of C. acnes useful herein can be obtained from public microorganism collections maintained for example by Leibniz Institute DSMZ-German Collection of Microorganisms and Cell Cultures (Inhoffenstr. 7B, D-38124 Braunschweig, Germany, https: www.dsmz.de/catalogues.html), or by American Type Culture Collection (ATCC) 10801 University Blvd. Manassas, Virginia 20110-2209, USA) or by European Collection of Cell Cultures (ECACC) (Heath Protection Agency—Porton Down Salisbury, Wilthsire SP4 0JG, United Kingdom), including C. acnes strain KPA171202 (DSMZ acc. No. DSM-16379), C. acnes subsp. defendens strain (acc. no. ATCC 11828), C. acnes strain (acc. no. ATCC 6919), C. acnes subsp. elongatum strain (ECACC acc. no. NCTC 13655), and others.

Further, C. acnes strains of virtually any type or subtype useful herein can be readily sampled and isolated from the skin of adult humans, preferably healthy adults, or from C. acnes biofilms on infected orthopedic prostheses. Any available protocol for isolation, culture and expansion of C. acnes can be adopted. Hence, in certain embodiments, a composition can be formed by isolating and culturing one or more C. acnes from a donor subject, particularly a donor subject not having oxidative stress-associated skin disease and preferably a donor subject having overall healthy skin, and combining it with other one or more carriers to form the composition.

By means of example and without limitation, one suitable protocol for obtaining C. acnes isolates from the skin was described in Holmberg et al. (2009). Therein, an area of 3×3 cm of the forehead of a human subject was swabbed with a cotton-tipped swab, moist with sterile physiologic saline, and plated on blood agar (LabM Ltd, Heywood, Bury, United Kingdom) containing horse blood (4%). The plates were incubated under anaerobic conditions (10% H₂, 10% CO₂, 80% N₂) at 37° C. for 72 hours. Isolates were characterized as C. acnes using routine microbiological criteria, including characteristic macroscopic appearance (typical small colonies), Gram-staining characteristics, and the production of catalase and indole. Isolates were frozen at −80° C. in glycerol (50% v/v). The bacteria could be further cultivated in Bacto Brain Heart Infusion broth (BHI; Becton and Dickinson, Sparks, MD, USA) supplemented with glucose (0.5%) and kept in an anaerobic chamber for 72 h at 37° C. When performed on48 healthy individuals, this yielded 48 C. acnes isolates belonging to types IA, IB, II (see Table 2 of Homberg, et al., incorporated by reference herein).

C. acnes cells can be further cultured and expanded in a variety of media, including rich or minimal media, preferably rich media such as those mentioned above. As would be understood by one of ordinary skill in the art, routine optimization would allow for use of a variety of types of media. Media can be supplemented with various additional components, including sugar sources. Some non-limiting examples of supplemental components include glucose, amino acids, vitamins, lipids, glycerol, and ATCC Trace Mineral Supplement. Similarly, other aspects of the medium, and growth conditions of the cells of the invention can be optimized through routine experimentation. For example, pH, temperature, and concentration of components within the compositions are non-limiting examples of factors which can be optimized. Liquid and/or solid cultures used to grow C. acnes cells can be housed in any of the culture vessels known and used in the art. In some embodiments, the C. acnes strains are grown in batches. In some embodiments, the bacterial strains are grown in fermenters. In some embodiments, the compositions comprising the bacterial strains are packaged. In certain embodiments, compositions comprising the bacterial strains are packaged in enteral syringes or sachets. In certain embodiments, the bacterial strains may be freeze-dried.

It was previously reported that some strains of C. acnes are associated with, contribute to or are causative of acne, while other strains show no such connection to acne (Fitz-Gibbon et al. 2013; Lomholt et al., 2010). These authors also characterized certain genetic and metabolic determinants of the ability of C. acnes to contribute to acne. Similarly, WO 2018/073651 described metabolic assays by which pathogenic and non-pathogenic strains of C. acnes could be identified and selected based on their expression of active linoleic acid isomerase which specifically converts cis-9, cis-12 linoleic acid into trans-10, cis-12 linoleic acid. Accordingly, in certain embodiments, the C. acnes strain or strains as intended herein are not associated with, do not contribute to and are not causative of acne. Such C. acnes strains can be readily isolated from healthy human skin (as opposed to human skin affected by acne).

The term “strain” is well-understood as a low-level taxonomic rank used within a species. In microbiology, a strain may be typically defined operatively—a strain is made up to the descendants of a single isolation in pure culture and usually is made up of a succession of cultures ultimately derived from an initial single colony; or alternatively, as an isolate or group of isolates that can be distinguished from other isolates of the same genus and species by phenotypic characteristics or genotypic characteristics or both. As used in the practice of the present invention, the term “strain” may be synonymous with the operative terms “isolate” or “clone”.

The term “live” as used herein is synonymous with “viable” and refers to any living intact state of a microorganism, such as active growth or dormancy, from which state it can multiply and/or reproduce itself in a medium capable of supporting the growth of the microorganism. Typically, substantially all C. acnes bacteria comprised by the compositions intended herein may be live or viable. For example, at least 50%, preferably at least 60%, more preferably at least 75%, still more preferably at least 90%, such as at least 95%, 96%, 97%, 98%, 99% or 100% of the bacteria in the composition are viable, such as capable of forming colonies when plated on a suitable solid medium.

C. acnes strains occur on the skin of most people. C. acnes strains can be pathogenic or non-pathogenic. As used herein, “pathogenic” C. acnes strains are strains that are associated with acne. Assays for the identification and selection of pathogenic and non-pathogenic C. acnes strains are described in WO 2018/073651.

C. acnes has been shown to comprise several distinct, major phylogenetic groups classified as types I, II and III, with the major type I clade being further divided into sub-clades known as types IA, IB and IC (Lomholt and Kilian, 2010). Sub-clade IA has been further subdivided into IA1 and IA2 (McDowell et al., 2012). Preferably, the at least one strain of the species C. acnes is a non-pathogenic strain of C. acnes. A genetic analysis of C. acnes strains revealed that strains which are non-pathogenic and not associated with acne are mainly members of (i) clade I, sub-clade IA, (ii) clade I, sub-clade IB and (ii) clade II. C. acnes strains belonging to these claims may be preferably employed herein. Accordingly, in one embodiment of the invention, the at least one strain of the species C. acnes belongs either to one of sub-clades IA, IB of clade I or to clade II. In one embodiment, the at least one strain of the species C. acnes belongs to sub-clade IA. In another embodiment, the at least one strain of the species C. acnes belongs to sub-clade IB. In yet another embodiment, the at least one strain of the species C. acnes belongs to clade II. If more than one strain is used in the skin care composition or kit of parts of the present invention, it is preferred that strains from different clades or sub-clades are mixed with each other. For example, in one embodiment, the skin care composition or kit of parts comprises at least one strain from sub-clade IA and at least one strain from sub-clade IB. In another embodiment, the skin care composition or kit of parts comprises at least one strain from sub-clade IA and at least one strain from clade II. In yet another embodiment, the skin care composition comprises at least one strain from sub-clade IB and at least one strain from clade II. In some embodiments, live bacteria of the C. acnes strain with SLST type A1 belong to sub-clade IA. In some embodiments, live bacteria of the C. acnes strain with SLST type D1 belong to sub-clade IA. In some embodiments, live bacteria of the C. acnes strain with SLST type H1 belong to sub-clade IB. In some embodiments, live bacteria of the C. acnes strain with SLST type K8 belong to sub-clade II.

In other embodiments, the skin care composition or kit of parts may comprise a mixture of C. acnes strains that include one or more clade I strains and one or more clade II strains. While clade II strains, as indicated above, may be less pathogenic than clade I strains, these strains can also be slower-growing than clade I strains, and less likely to be able to colonize the skin on their own. Accordingly, it may in some embodiments be advantageous that the skin care composition or kit of parts includes a mixture of strains that include both clade I and clade II strains which allow for comparatively improved colonization of the skin by clade II strains.

Non-limiting examples for non-pathogenic strains of C. acnes include, but are not limited to, SLST type strains A1, D1, A5, C1, C3, H1, H2, H3, K1, K2, K4, K6, K8, K9, L1 and F4. It is particularly preferred that the skin care composition or kit of parts of the present invention includes at least one SLST type A1 strain and/or at least one SLST type D1 strain and/or at least one SLST type H1 strain and/or at least one SLST type K8 strain. In one embodiment, the skin care composition or kit of parts of the present invention includes at least one SLST A1 strain, and more preferably two or more SLST type A1 strains, such as, 3, 4, 5, 6, 7, 8, 9 or 10 SLST A1 strains. In another embodiment, the skin care composition or kit of parts of the present invention includes at least one SLST D1 strain, and more preferably two or more SLST type D1 strains, such as, 3, 4, 5, 6, 7, 8, 9 or 10 SLST D1 strains. In yet another embodiment, the skin care composition or kit of parts of the present invention includes at least one SLST H1 strain, and more preferably two or more SLST type H1 strains, such as, 3, 4, 5, 6, 7, 8, 9 or 10 SLST H1 strains. In yet another embodiment, the skin care composition or kit of parts of the present invention includes at least one SLST K8 strain, and more preferably two or more SLST type K8 strains, such as, 3, 4, 5, 6, 7, 8, 9 or 10 SLST K8 strains. In some other embodiments, the skin care composition or kit of parts of the present invention includes a combination of two or more of the SLST type A1, D1, H1 and K8 strains. Such as for example, in an embodiment the skin care composition or kit of parts of the present invention includes at least one SLST type H1 strain in combination with at least one SLST K8 strain. Or, in some embodiments, the skin care composition or kit or parts of the present invention includes at least one SLST type H1 strain in combination with at least one SLST K8 strain and in combination with at least one SLST type D1 strain. In some other embodiments, the skin care composition or kit of parts of the present invention includes at least one SLST type H1 strain in combination with at least one SLST D1 strain.

In a particularly preferred embodiment, the skin care composition or kit of parts of the present invention comprises at least one C. acnes strain selected from the group consisting of SLST type A1 strain, SLST type H1 strain, SLST type D1 strain and SLST type C. acnes K8 strain. Even further, the skin care composition or kit of parts as disclosed herein may comprise 2, 3 or all 4 of the SLST type A1 strain, SLST type H1 strain, SLST type D1 strain and SLST type C. acnes K8 strain.

As indicated above, the strains designation referred to herein is based on the single-locus sequence typing (SLST) scheme described in Scholz et al., 2014 using locus PPA2385 as the SLST target sequence. The SLST types can be interrogated using the following primers (Table 1):

TABLE 1
Primer sequences used to type bacterial colonies

| SEQ | | |
|---|---|---|
| ID | | |
| NO: | Description | Sequence |
| 1 | Forward | CAGCGGCGCTGCTAAGAACTT |
| | primer | |
| 2 | Reverse | CCGGCTGGCAAATGAGGCAT |
| | primer | |
| 3 | SLST- | TCGTCGGCAGCGTCAGATGTGTATAAG |
| | Adapter-FW | AGACAGCAGCGGCGCTGCTAAGAACTT |
| 4 | SLST- | GTCTCGTGGGCTCGGAGATGTGTATAA |
| | Adapter-RV | GAGACAGCCGGCTGGCAAATGAGGCAT |

The sequences of the PPA2385 locus of the different strains identified by Scholz are listed as SEQ ID NO: 1-76 in Table 1 on p. 20-31 of WO 2018/073651, incorporated by reference herein.

Accordingly, by means of illustration, a “SLST type A1” strain is a strain that comprises in its genome a sequence of the PPA2385 locus which is 100% identical to the following sequence:

(SEQ ID NO: 5)
GTTGCACACCAGGGGGTCAACTTGGCGTCCTCAGTTCAAAATTGA
TTCAAACTAACAGTTCCATGTCGGGAAACAGCACCAGGAAGCTCG
TGACATATCGTCTTTCATTGCGAGAAACATCTTACTTATGTACAT
TTCTAAGCTATAGCGTCTACCCTTGTCAGACCCAGGACGATGGGT
GTCACATCTCCTTTCTAGTCAACCTAAGAGAGGAGGAAATGCCGC
GATATATGTTCCACCCTGTCATCACGAAGGCCACCACAATCTATC
CCAGAACAGCCGGCACTTCACTCACGATGCCCCGATGCTGGATTC
CTATTGTCGCCCTTATTAGGGCAAGCGGTGCCAGTAGCAGAATAT
GTCACCTCAACAACTCGATCCACCCCTGCCCATTACATGGGTAAC
ATATCCATGGAGGTTCGATGTATACTCGAGGATACAGTCGTCCAT
CACGCCCGCCTACATACCCATTACATCAGCATAG.

A “SLST type D1” strain is a strain that comprises in its genome a sequence of the PPA2385 locus which is 100% identical to the following sequence:

(SEQ ID NO: 6)
GTTGCACACCAGGGGGTCAACTTGGCGTCCTCAGTTCAAAATTGA
TTCAAACTAACAGTTCCATGTCGGGAAACAGCACCAGGAAACTCG
TGACATATCGTCTTTCATTGCGAGAAACATCTTACTTATGTACAT
TTCTAAGCTATAGCGTCTACCCTTGTCAGACCCAGGACGATGGGT
GTCACATCCCCTTTCTAGTCAACCTAAGAGAGGAGGAAATGCCGC
GATATATGTTCCGCCCTGTCATCACGAAGACCACCACAATCTATC
CCAGAACAGCCGGCACTTCACTCACGATGCCCCGATGCTGGATTC
CTATTGTCGCCCTTATTAGGGCAAGCGGTGCCAGTAGCAGAATAT
GTCACCTCAACAACTCGATCCACCCCTGCCCATTACATGGGTAAC
ATATCCATGGAGGTTCGATGTATACTTGAGGATACAGTCGTCCAT
CACGCCCACCTACATACCCATTACATCAGCATAG.

A “SLST type H1” strain is a strain that comprises in its genome a sequence of the PPA2385 locus which is 100% identical to the following sequence:

(SEQ ID NO: 7)
GTTGCACACCAGGGGGTCAACTTGGCGTCCTCAGTTCAAAATTGA
TTCAAACTAACAGTTCCATGTCGGGAAACAGCACCAGGAAGCTCG
TGACATATCGTCTTTCATTGCGAGAAACATCTTACTTATGTACAT
TTCTAAGCTATATCGTCTACCCTTGTCAGACCCAGGACGATGGAT
GTCACATCCCCTTTCTAGTCAACCTAAGAGAGGAGGAAATGCCGC
GATATATGTTCCACCCTGTCATCACGAAGGCCACCACAATCTATC
CCAGAACAGCCGGCACTTCACTCACGATGCCCCGATGCTGGATTC
CTATTGTCGCCCTTATTAGGGCAAGCGGTGCCAGTAGCAGAATAT
GTCACCTCAACAACTCGATCCACCCCTGCCCATTACATGGGTAAC
ATATCCATGGAGGTTCGATGTATATTCGAGGATACAGTCGTCCAT
CACGCCCGCCTACATACCCATTACATCAGCATAG.

A “SLST type K8” strain is a strain that comprises in its genome a sequence of the PPA2385 locus which is 100% identical to the following sequence:

(SEQ ID NO: 8)
ATTGCACACCAGGGGGTCAACTTGGTGTCCTCAGTTCAAAATTGG
TTCAAACTAACGGTTCCGTGTCGGGAAACAGCACCAGAAAACTCG
TGACATATCGTCTTTCATTGCGAGAAACATCTTACTTATACACAT
TTCTAAGCTATATTGTCTACCCCTGTCAGACCCAGGACGATGGGT
GTCATATCCCCTTTCCAGTCAACCTAAGAAGGGAGGAAATGCCGC
GATATATGTTCCGCCCTGTCATCATGAATGCCACCACAATCTATC
CCGGAACAGCCGTACTTCACCCACCATGCCCCGATGCTGGATTCC
TATTGTCGCCCTTATTAGAGCAAGCGGTGCCAGCAGCAGAATATT
TCACCTCAGCAACTCGATCCGCTCCTGCCCATTACATGGGTAACA
TATCCATGGAGGTACGATGTATGCATCGAGGATGCAGTCGTCTAC
TATGCCCGCCTACATACCCATTCCATCAGCATAG

The SLST scheme is also described in more detail in WO 2018/073651. Sequence identification of the PPA2385 locus can be performed as described in WO 2018/073651 by PCR amplification and DNA sequencing using the nucleotide primer set forth as SEQ ID NO: 77-82 in Table 2 on p. 31 of WO 2018/073651.

The composition or kit of parts of the present invention may include both pathogenic strains and non-pathogenic strains of C. acnes. However, in a preferred embodiment of the invention, the skin care composition or kit of parts comprises exclusively non-pathogenic strains of C. acnes. It is particularly preferred that the skin care composition or kit of parts of the invention does not include a ribotype 6 (RT6) strain of C. acnes. The ribotype classification system is based on difference in the 16S rDNA sequence between different strains of C. acnes. The ribotype system is explained, for example, in Fitz-Gibbon et al. 2013. It is further particularly preferred that the skin care composition or the kit of parts of the invention does not include a Phylotype III strain of C. acnes.

C. acnes strains are normally able to produce the signaling molecule trans-10, cis-12 linoleic acid from its precursor molecule linoleic acid, the latter of which is naturally present in the sebum. Trans-10, cis-12 linoleic acid is thought to stimulate sebum production and secretion which is important for C. acnes colonization of the skin. In this way, trans-10, cis-12 linoleic acid promotes the onset of acne. Dependent on the skin of a subject, it may be favorable to either reduce or increase sebum production. For example, it may be useful to reduce sebum production in a skin of a subject suffering from acne or oily skin. To the contrary, it may be useful to increase sebum production in the skin of a subject suffering from dry skin or aged skin.

In one embodiment, the one or more C. acnes strains of the present invention are hence selected based on its ability to produce trans-10, cis-12 linoleic acid. In one preferred embodiment, strains that produce low levels of trans-10, cis-12 linoleic acid are selected for use against acne or oily skin. Without wishing to be bound by theory, these strains are thought to reduce sebum production, which is useful for preventing or reducing the symptoms of acne or oily skin. SLST type strains C3, C1, F4, A5, K1, K2, K8 and L1 produce only low amounts of trans-10, cis-12 linoleic acid. In another preferred embodiment, strains that produce high levels of trans-10, cis-12 linoleic acid are selected for use against dry skin. Without wishing to be bound by theory, these strains are thought to increase sebum production, which is useful for preventing or reducing the symptoms of dry skin. SLST type strain A1 produces high amounts of trans-10, cis-12 linoleic acid. In one embodiment, the one or more C. acnes strains to be administered to the skin area have been isolated from the skin microbiome of a donor subject. The subject may not be afflicted with acne or oily skin or may suffer from mild, moderate or severe acne. In another embodiment, the strains that have been isolated from the skin microbiome of a donor subject are non-pathogenic strains.

The one or more live C. acnes may also comprise one or more genetically modified strains of C. acnes. In another embodiment, one or more genetically C. acnes strain may be combined with one or more naturally occurring strains of C. acnes. The genetically modified strains have preferably been modified to produce lower or higher amounts of trans-10, cis-12 linoleic acid. The production of trans-10, cis-12 linoleic acid can be detected as described in U.S. Pat. No. 6,743,609 or by other commonly known methods, such as FAME (fatty acid methyl esters) or gas chromatography. In certain other embodiments, the one or more live C. acnes strains may only comprise naturally occurring strains of C. acnes, i.e., only C. acnes strain(s) that have not been genetically modified by man.

In some embodiments, each of the C. acnes strains in the skin care composition is present in an amount of 1.0×10⁴-1.0×10¹¹ colony forming units per ml (CFU/ml), preferably 1.0×10⁴-1.0×10⁹ CFU/ml, even more preferably 1.0×10⁶-1.0×10⁹ CFU/ml such as 1.0×10⁷-1.0×10⁹ CFU/ml, or 1.0×10⁸-1.0×10⁹ CFU/ml. For example, the at least one C. acnes strain may be present in an amount of at least 1.0×10⁵ CFU/ml, preferably at least 1.0×10⁶ CFU/ml, more preferably at least 1.0×10⁷ CFU/ml, such as at least 1.0×10⁸ CFU/ml, or at least 1.0×10⁹ CFU/ml of the skin care composition.

In some embodiments, each of the C. acnes strains that is present in the skin care composition is present in an amount of 1.0×10⁴-1.0×10¹¹ CFU/ml, preferably 1.0×10⁴-1.0×10⁹ CFU/ml, more preferably 1.0×10⁶-1.0×10⁹ CFU/ml and even more preferably 1.0×10⁷-1.0×10⁹ CFU/ml, or 1.0×10⁸-1.0×10⁹ CFU/ml. For example, each of the C. acnes strains may be present in an amount of at least 1.0×10⁵ CFU/ml, preferably at least 1.0×10⁶ CFU/ml, more preferably at least 1.0×10⁷ CFU/ml, such as at least 1.0×10⁸ CFU/ml, or at least 1.0×10⁹ CFU/ml of the composition. For example, if the composition of the present invention comprises one SLST type H1 strain and one SLST type K8 strain, each of these strains may be present in an amount of 1.0×10⁴-1.0×10⁹ CFU/ml, such as 1.0×10⁹ CFU/ml, 2.0×10⁹ CFU/ml, 3.0×10⁹ CFU/ml, 4.0×10⁹ CFU/ml, 5.0×10⁹ CFU/ml, 6.0×10⁹ CFU/ml, 7.0×10⁹ CFU/ml, 8.0×10⁹ CFU/ml, or 9.0×10⁹ CFU/ml.

In some embodiments, the overall amount of (optionally lyophilized or spray-dried) bacteria in the composition is 1.0×10⁴-1.0×10¹¹ CFU/ml, such as 1.0×10⁴-1.0×10⁹ CFU/ml, more preferably 1.0×10⁵-1.0×10¹⁰ CFU/ml, and even more preferably 1.0×10⁷-1.0×10¹⁰ CFU/ml, or 1.0×10⁸-1.0×10⁹ CFU/ml. For example, the bacteria may be collectively present in the composition in an amount of at least 1.0×10⁵ CFU/ml, preferably at least 1.0×10⁶ CFU/ml, more preferably at least 1.0×10⁷ CFU/ml, such as at least 1.0×10⁸ CFU/ml, at least 1.0×10⁹ CFU/ml, or at least 1.0×10¹⁰ CFU/ml of the composition. It is particularly preferred that the bacteria are collectively present in the composition in an amount of at least 1.0×10¹⁰ CFU/ml, 2.0×10¹⁰ CFU/ml, 3.0×10¹⁰ CFU/ml, 4.0×10¹⁰ CFU/ml, 5.0×10¹⁰ CFU/ml, 6.0×10¹⁰ CFU/ml, 7.0×10¹⁰ CFU/ml, 8.0×10¹⁰ CFU/ml, or 9.0×10¹⁰ CFU/ml of the composition. One of ordinary skill in the art will be readily able to determine the amount of bacteria in the composition.

In some embodiments, the skin care composition is an aqueous preparation, such as a gel. Aqueous preparations as intended herein encompass aqueous solutions, as well as aqueous dispersions. In one embodiment, the composition is an oil-in-water emulsion. If the composition contains an oil phase, e.g. when using an oil-in-water emulsion, it is preferred that the oil phase contains triglycerides and/or octyldodecanol. In addition, the oil phase may contain one or more oils selected from the group of lecithin, olive oil, sunflower oil, jojoba oil, soya oil, peanut oil, rapeseed oil, almon oil, palm oil, coconut oil, castor oil, wheat germ oil, grape seed oil, safflower oil, evening primrose oil, macadamia nut oil and the like.

In some embodiments, the composition comprising one or more live C. acnes strain and the one or more compounds selected from the group consisting of a polyethylene glycol ester of a fatty acid, glycerol, sorbitol, lactic acid or a salt thereof, and cetearyl sulphate is a water-in-oil emulsion.

In other embodiments, the composition comprising one or more live C. acnes strain and one or more compounds selected from the group consisting of a polyethylene glycol ester of a fatty acid, glycerol, sorbitol, lactic acid or a salt thereof, and cetearyl sulphate can be an ointment or a cream. In the context of the present invention, an ointment is a semisolid preparation containing an ointment base and optionally one or more active agents, more specific the one or more live C. acnes strain and one or more of said compounds. Examples of suitable ointment bases include hydrocarbon base (e.g., petrolatum, white petrolatum, yellow ointment, and mineral oil), absorption bases (e.g., hydrophilic petrolatum, anhydrous lanolin, lanolin, and cold cream), water-removable bases (e.g., polyethylene glycol ointments).

In still other embodiments, the composition as disclosed herein can be a lotion. A lotion is a low- to medium-viscosity liquid formulation. A lotion can contain finely powdered substances that are insoluble in the dispersion medium through the use of suspending agents and dispersing agents. Alternatively, a lotion can have the dispersed phase liquid substances that are immiscible with the vehicle and are usually dispersed by means of emulsifying agents or other suitable stabilizers.

In certain embodiments, the compositions may be comprised by a mask, pad, patch, a two-chamber device (two-component dispensing system), or make-up. The present compositions may typically be intended as ‘leave-on’ compositions. By means of an example and without limitation, the composition may comprise one or more components provided in one container, such as one chamber of a multiple-chamber device (e.g., a two-chamber dispensing system), and one or more components provided in another container, such as another chamber of the multiple-chamber device. Such arrangement allows the consumer or practitioner to admix the components of the composition shortly before use. For example, the kit of parts may comprise the one or more live C. acnes strain or strains in one container, such as one chamber of a multiple-chamber device (e.g., a two-chamber dispensing system), and a composition comprising the polyethylene glycol ester of a fatty acid or comprising one or more compounds selected from the group consisting of a polyethylene glycol ester of a fatty acid, glycerol, sorbitol, lactic acid or a salt thereof, and cetearyl sulphate in another container, such as another chamber of the multiple-chamber device, to be admixed by the consumer or practitioner before use. Multiple-chamber (such as two- or dual-chamber) dispensers, such as bottles, tubes, jars, pumps, etc. are generally known and commercially available. By means of an example and not limitation, WO 2012/153206, WO 2017/168263, and WO 2018/060799 by Bormioli Rocco SPA (Fidenza, Italy) describe systems, marketed inter alia as the New Shaker® single-dose dispenser, comprising a container and a closure capsule for closing the container, wherein the closure capsule includes an enclosure defined at least partly by a frangible bottom or mouth. Changing the configuration of the frangible bottom or mouth from intact to open allows to mix the previously separated contents of the container and the enclosure directly before use. In certain embodiments, the one or more live C. acnes strain or strains may be in a lyophilised form. Typically, such form is itself a composition comprising the one or more live C. acnes strain or strains and components of a suitable bacterial lyophilisation medium.

In some embodiments, the one or more live C. acnes strain are provided as a lyophilizate which is suspended in oil and is provided in a first container. A second container contains a composition comprising a polyethylene glycol ester of a fatty acid. This composition can be provided as a carrier gel, cream, ointment or lotion. The content of both containers is mixed in the hands of the user immediately prior to applying the mixture to the face. The second container may also contain one or more compounds selected from the group consisting of a polyethylene glycol ester of a fatty acid, glycerol, sorbitol, lactic acid or a salt thereof, and cetearyl sulphate.

The skin care composition or the composition comprising a polyethylene glycol ester of a fatty acid in the kit of parts as taught herein may further comprise one or more carriers or excipients, which can broadly include any and all solvents, diluents, buffers (such as, e.g., neutral buffered saline, phosphate buffered saline, or optionally Tris-HCl, acetate or phosphate buffers), solubilizers (such as, e.g., Tween® 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatizers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for the formulation of pharmaceutical and cosmetic compositions is well known in the art. In certain embodiments, the compositions may further comprise hydrators, exfoliants, humectants, emollients, and/or synthetic surfactants.

In some embodiments, the skin care composition or the kit of parts, such as the composition comprised by the kit of parts, further comprises an emollient. As used herein, an emollient is a compound that moisturizes and/or softens the skin. Emollients normally reduce the roughness, cracking and/or irritation of the skin by penetrating into deeper layers of the skin. Emollients commonly used in skin care products comprise plant oils, like sesame oil, coconut oil, olive oil, almond oil, macadamia nut oil, cottonseed oil, or peanut oil, silicone oils, like dimethylpolysiloxane and cyclomethicone, fatty acids, and fatty alcohol ethers. The emollient can for example be selected from dicaprylyl carbonate, ethylhexyl cocoate, and mixtures thereof. When dicaprylyl carbonate is used as an emollient, it is preferably used in the final skin care composition in an amount of 0.05 to 25.0 wt %, more preferably 2.0 to 20.0 wt %, and more preferably 5.0 to 10.0 wt % or 7.5 to 10.0 wt %, relative to the total weight (w/w) of the composition. Stated differently, the amount of dicaprylyl carbonate in the skin care composition of the invention may be at least 0.05 wt %, at least 0.1 wt %, at least 0.25 wt %, at least 0.5 wt %, at least 0.75 wt %, at least 1.0 wt %, at least 1.25 wt %, at least 1.5 wt %, at least 1.75 wt %, at least 2.0 wt %, at least 2.5 wt %, at least 3 wt %, at least 4.0 wt %, at least 5.0 wt %, at least 6.0 wt %, at least 7.0 wt %, at least 8.0 wt %, or at least 9.0 wt %, relative to the total weight (w/w) of the skin care composition. When ethylhexyl cocoate is used as an emollient, it is preferably used in the final skin care composition in an amount of 0.05 to 25.0 wt %, more preferably 2.0 to 20.0 wt %, and more preferably 5.0 to 10.0 wt % or 7.5 to 10.0 wt %, relative to the total weight (w/w) of the composition. Stated differently, the amount of ethylhexyl cocoate in the skin care composition of the invention may be at least 0.05 wt %, at least 0.1 wt %, at least 0.25 wt %, at least 0.5 wt %, at least 0.75 wt %, at least 1.0 wt %, at least 1.25 wt %, at least 1.5 wt %, at least 1.75 wt %, at least 2.0 wt %, at least 2.5 wt %, at least 3 wt %, at least 4.0 wt %, at least 5.0 wt %, at least 6.0 wt %, at least 7.0 wt %, at least 8.0 wt %, or at least 9.0 wt %, relative to the total weight (w/w) of the skin care composition. When dicaprylyl carbonate and ethylhexyl cocoate are used in combination with each other as emollients, it is preferred that the overall amount of emollient is at least 0.05 wt %, but does not exceed 20.0 wt %, more preferably does not exceed 15.0 wt % or 10.0 wt % relative to the total weight (w/w) of the composition.

In another embodiment, the skin care composition or the kit of parts, such as the composition comprised by the kit of parts, further comprises a thickener. Thickeners are compounds that increase the viscosity of a cosmetic or pharmaceutical formulation. Thickeners are often polymers that absorb water and swell up, thereby making the composition more viscous. Thickeners commonly used in skin care products comprise bean gum, xanthan gum, gelatine, Carbauba wax, and stearic acid.

In another embodiment, the skin care composition or the kit of parts, such as the composition comprised by the kit of parts, further comprises a pH adjuster. Since the composition of the invention is used on the human skin, it will preferably have a neutral or slightly acidic pH to make it more compatible with the acidic environment of the skin. The composition may have a pH in the range from about 2.5 to about 7.5, preferably from about 4.0 to about 7.0, and more preferably from about 6.0 to about 7.5, preferably from about 4.0 to about 7.0, and more preferably from about 6.0 to about 7.0. An acidic pH in a cosmetic or pharmaceutical formulation can be normally achieved by adding an acid, such as formic acid, acetic acid, butyric acid, valeric acid, caproic acid, enanthic acid, or caprylic acid. Preferably, the pH adjuster is a citric acid/citrate buffer, which tends to display less interference with or can even promote the ability of the bacteria in the composition to grow and replicate after administration to the skin.

In yet another embodiment, the skin care composition or the kit of parts, such as the composition comprised by the kit of parts, further comprises a filler. As used herein, a filler is a compound that aids in making the skin care composition more homogeneous by uniformly dispersing in the composition. Fillers are used to improve the sensory properties of the skin. Depending on the filler material, the end product may confer a silky, dry, smooth, or powdery skin feel. Fillers that can be used in the present invention can for example be distarch phosphate, tapioca starch, and mixtures thereof.

In another embodiment, the skin care composition or the kit of parts, such as the composition comprised by the kit of parts, further comprises a solubilizer. As used herein, a solubilizer is a compound that aids in the solubilization of hydrophobic substances in aqueous and alcohol formulations. For example, a solubilizer may render feasible the solubilization of perfume oils and other hydrophobic substances, such as vitamins, into aqueous composition. A particularly preferred solubilizer is polyethylene glycol (PEG-)40 castor oil, which tends to display less interference with or can even promote the ability of the bacteria in the composition to grow and replicate after administration to the skin.

Surfactants can be selected from the group consisting of anionic, cationic, amphoteric and alkylglycosidic surface active agents. Suitable anionic surfactants include, for example, alkyl and alkyl ether sulfates (such as sodium cocoalkyl triethylene glycol ether sulfate); water-soluble salts, phosphates such as monoalkyl, dialkyl, and trialkylphosphate salts form by the reaction of phosphorous pentoxide with monohydric branched or unbranched alcohols having from about 8 to about 24 carbon atoms (such as mono or dilaurylphosphate); the reaction products of fatty acids esterified with isethionic acid and neutralized with an alkaline reagent; sulfonated fatty acids (such as alpha sulphonated coconut fatty acid and lauryl methyl ester); acyl isethionates (such as ammonium cocoyl isethionate, sodium cocoyl isethionate, sodium lauroyl isethionate); acyl glutamates (such as sodium lauroyl glutamate and sodium cocoyl glutamate); sulfocuccinate salts (such as disodium N-octadecylsulfosuccinamate and sodium dioctyl sulfosuccinate); carboxylates, including alkyl ether carboxylates (such as sodium laureth carboxylate); acyl lactylates (such as sodium cocoyl lactylate); alanoyl sarcosinates (such as sodium lauroyl sarcosinate, sodium cocoyl sarcosinate, and ammonium lauroyl sarcosinate); alkylglyceryl ether sulfonates (such as sodium cocoglyceryl ether sulfonate); and olefin sulfonates (such as sodium C₁₄₋₁₆ olefin sulfonates). Suitable non-ionic surfactants include, but are not limited to, for example, compounds produced by the condensation of alkylene oxide with an organic hydrophobic compound which can be either aliphatic, alicyclic or aromatic in structure. Non-ionic surfactants are illustrated by polyethylene oxide condensates of C6-C12 alkylphenols; condensates of ethylene oxide with the reaction product of propylene oxide and ethylenediamine; long chain tertiary amine oxides; long chain tertiary phosphine oxides; long chain dialkyl sulfoxides; and the like. Suitable cationic surfactants are compounds containing positively charged amine or quaternary ammonium groups. Suitable amphoteric surfactants include derivatives of aliphatic quaternary ammonium, phosphonium and sulfonium compounds. One class of amphoteric surfactants are zwitterionic compounds such as betaines, sultaines and phosphobetaines. Illustrative of a betaine is cocoamidopropyl betaine. Another class of amphoteric surfactants are compounds containing an amine group and an anionic group such as carboxylate, sulfonate, sulfate, phosphate or phosphonate, as illustrated by sodium 3-dodecylaminopropionate and sodium 3-dodecylaminopropane sulfonate.

In a particular embodiment, the cosmetic composition comprises at least one of the following: sodium cocoamphoacetate, propylene glycol, sodium laureth sulfate, citric acid, sodium benzoate, salicyl acid.

The skin care compositions or kit of parts, such as the composition comprised by the kit of parts, may include, apart from the above components, commonly known excipients including perfumes, pigments, colorants, dyes, waxes, masking agents, stabilizers, sunscreens, emulsifiers, medicaments, antiseptics, chelating agents, protectants, viscosifiers, vitamins, panthenol, ubiquinone Q10, hyaluronic acid, or any combinations thereof.

The skin care composition or kit of parts described herein are useful for the modulation of the skin microbiome in a subject, and in particular for maintaining or restoring a healthy or youthful skin, such as skin that is free of acne or wrinkles. The composition or kit of parts of the present invention can help the skin to revert microbiome disease states to healthy microbiome states in a subject. More specifically, with the present compositions and kit .of parts, the growth of the at least one C. acnes strain on the skin is stimulated and boosted, resulting in beneficial effects on the skin. It is particularly preferred that the subject is a human.

The term “subject” typically and preferably denotes humans, but may also encompass reference to non-human animals, preferably warm-blooded animals, even more preferably mammals, such as, e.g., non-human primates, rodents, canines, felines, equines, ovines, porcines, etc. The term “non-human animals” includes all vertebrates, e.g., mammals, such as non-human primates, particularly higher primates, sheep, dogs, rodents (e.g., mice or rats), guinea pigs, goats, pigs, cats, rabbits, cows, and non-mammals such as chickens, amphibians, reptiles etc. In certain embodiments, the subject is a non-human animal. In certain embodiments, the subject is a non-human mammal. In certain embodiments, the subject is a transgenic non-human animal or non-human mammal. In preferred embodiments, the subject is human. In other embodiments, the subject is an experimental animal or animal disease model. The term does not denote a particular age or sex, and includes inter alia newborns, children, adolescents, and adults including the elderly, whether male or female.

In aspects and embodiments relating to therapeutic or prophylactic interventions the terms “subject” and “patient” may be used interchangeably. Suitable subjects or patients in need of preventing or treating a disease as taught herein include those that would benefit from treating the disease or those in whom said disease is to be prevented. Such subjects or patients include without limitation patients presenting to a physician for a screening for the disease, patients presenting to a physician with symptoms and signs indicative of the disease, patients diagnosed with the disease, patients prone to contract or develop the disease, patients who have received or are undergoing treatment of the disease, and patients having a disease as taught herein in remission. In aspects and embodiments relating to cosmetic treatments, the term “subject” which does not imply the presence of a pathological condition in the subject, may be preferred over the term “patient”. A phrase such as “a subject in need of” a certain intervention, such as the treatment of a given condition, includes subjects that would benefit from the treatment of that condition. The presence or absence of a need for the subject to receive a given intervention such as treatment may be inferred by various diagnostic or benefit-evaluation methods.

Methods for treating the skin of a subject by administering a skin care composition as described hereinabove are also provided, in a particular a skin care composition comprising live bacteria of at least one C. acnes strain and an ester of a polyethylene glycol and a fatty acid. These methods may be cosmetic or therapeutic methods. In one aspect of the invention, a method of treating or preventing a disease or condition selected from the group consisting of acne oily skin, progressive macular hypomelanosis, dandruff, atopic eczema, atopic dermatitis and rosacea in a subject, said method comprising the topical administration of a skin care composition described herein above. In another aspect, the invention provides a skin care composition or kit of parts as disclosed herein for use in the treatment and/or prevention of a disease or condition selected from the group consisting of acne oily skin, progressive macular hypomelanosis, dandruff, atopic eczema, atopic dermatitis, and rosacea. In preferred embodiments, the skin care composition or the kit of parts as disclosed herein is for use in the treatment and/or prevention of acne. In another preferred embodiment, the skin care composition or kit of parts is for use in the prevention of reoccurrence of acne in a subject who has received a standard acne treatment. It is particularly preferred that the subject is a human.

In another aspect, a method is provided for treating or preventing an oxidative stress-associated skin disease in a subject, said method comprising the topical administration of a skin care composition as disclosed herein, to an area of the subject's skin. In another aspect, the skin care composition or kit of parts as disclosed hereinabove are provided for use in the treatment of an oxidative stress-associated skin disease in a subject. Preferably, the oxidative stress-associated skin disease is selected from actinic keratosis (AK), basal cell carcinoma (BCC), squamous cell carcinoma (SCC), dandruff, seborrheic dermatitis, acne, inflammation, dermatitis, psoriasis, eczema, rosacea, urticaria and vitiligo. In certain preferred embodiments, the oxidative stress-associated skin disease is AK. In further preferred embodiments, the oxidative stress-associated skin disease is BCC. In yet further preferred embodiments, the oxidative stress-associated skin disease is SCC. In yet further preferred embodiments, the oxidative stress-associated skin disease is dandruff. In yet further preferred embodiments, the oxidative stress-associated skin disease is seborrheic dermatitis.

Any such diseases may include components, aspects or processes other than those related to the oxidative stress. In certain embodiments, the present prophylactic uses or methods treat, address or impinge on the oxidative stress component(s), aspect(s) or process(es) of any such disease, for example, they improve or restore the redox balance in the skin tissue or cells.

In another aspect, a method for improving the appearance of the skin of a subject is disclosed, said method comprising the topical administration of the skin care composition as disclosed herein above, to an area of the subject's skin. In another aspect, a method for modulating the sebum production of skin cells of a subject is provided, said method comprising the topical administration of the skin care composition as disclosed herein above, to an area of the subject's skin. In still another aspect, a method for maintaining a healthy or youthful appearance of the skin in a subject is disclosed, said method comprising the topical administration of the skin care composition as disclosed herein to an area of the subject's skin.

In another aspect, a method is provided for stimulating or boosting the growth of at least one endogenous C. acnes strain on the skin of a subject, said method comprising the topical administration of a skin care composition as disclosed herein above to an area of the subject's skin.

In still another aspect, a method for stimulating or boosting the growth of at least one C. acnes bacterial strain in vitro is provided. Said method comprises administration of a composition comprising an ester of a polyethylene glycol and a fatty acid to at least one C. acnes bacterial strain as disclosed herein in vitro. In some embodiments, the fatty acid is a saturated fatty acid. In some embodiments, the fatty acid is a C₁₆-C₁₈ fatty acid. In some further embodiments, the fatty acid is a saturated C₁₆-C₁₈ fatty acid, such as a stearic acid.

Further provided is a method for stimulating or boosting the growth of at least one C. acnes bacterial strain in vitro, wherein the method comprises administration of a composition comprising one or more compounds selected from the group consisting of a polyethylene glycol ester of a fatty acid, glycerol, sorbitol, lactic acid or a salt thereof, and cetearyl sulphate to at least one C. acnes bacterial strain as disclosed herein in vitro. In certain embodiments, the composition may comprise any mixture of two or more of said compounds.

The cosmetic uses or methods as taught herein generally comprise administering to the skin of the subject a cosmetically effective amount of the skin care composition as taught herein, that is an amount sufficient to elicit the cosmetic effect relating to the prevention or reduction of skin aging in the subject, that is being sought by the cosmetician or beautician, in either a single or multiple doses. The prophylactic or therapeutic uses or methods as taught herein generally comprise administering to the skin of the subject a prophylactically or therapeutically effective amount of the skin care composition as taught herein. The term “therapeutically effective amount” generally denotes an amount sufficient to elicit the pharmacological effect or medicinal response in a subject that is being sought by a medical practitioner such as a medical doctor, clinician, surgeon, veterinarian, or researcher, which may include inter alia alleviation of the symptoms of the disease being treated, in either a single or multiple doses. The term “prophylactically effective amount” generally denotes an amount sufficient to elicit the preventative effect, such as inhibition or delay of the onset of a disease, in a subject that is being sought by the medical practitioner, in either a single or multiple doses. Appropriate cosmetically effective doses of the present compositions may be determined by a cosmetician with due regard to the age and skin condition of the patient. Appropriate prophylactically or therapeutically effective doses of the present compositions may be determined by a qualified physician with due regard to the nature and severity of the disease, and the age and condition of the patient. The effective amount of the compositions described herein to be administered can depend on may different factors and can be determined by one of ordinary skill in the art through routine experimentation. Several non-limiting factors that might be considered include biological activity of the active ingredient, nature of the active ingredient, characteristics of the subject to be treated, etc.

The term “to administer” generally means to dispense or to apply, and typically includes both in vivo administration and ex vivo administration to a tissue, preferably in vivo administration. Generally, compositions may be administered systemically or locally. Given the nature of the present cosmetic, prophylactic or therapeutic treatments, and the nature of the active ingredient, the present compositions may be preferably configured for topical administration to the skin of the subject.

The term “topical administration” as generally used in the cosmetic and medical fields denotes the application of compositions directly to a part of the body. Particularly in the present case the term refers to topical administration onto the skin of a subject, more particularly onto the surface of the skin of the subject, and even more particularly onto a part, region or area of the surface of the subject's skin where the cosmetic or pharmacological effect is sought. Topical administration is typically not intended to and does not elicit any systemic effects.

When the compositions are applied to the skin it is preferred that the amount of the composition applied to the skin is between 0.5 g and 2.0 g, more preferably between 0.5 g and 1.0 g. Stated differently, the amount of the composition may correspond to at least 1.0×10⁵ CFU, at least 1.0×10⁶ CFU, at least 1.0×10⁷ CFU, at least 1.5×10⁷ CFU, at least 2.0×10⁷ CFU or at least 2.5×10⁷ CFU.

The skin care composition of the present invention may be provided as ready-to-use composition which is suitable for direct topical administration to the skin. Such a composition may be provided in different forms, including, but not limited to, in the form of a gel, cream, lotion, ointment, past, soft, paste, suspension, solution, salve, wax, milk, emulsion, or the like. In such a composition, the (optionally lyophilized or spray-dried) live bacteria will be present in the admixture with other cosmetic or pharmaceutical excipients described elsewhere herein, such as emollients, fillers, and the like. Upon application of these compositions to the skin, the dried bacteria will be re-activated on the skin of the subject to which the product is applied. Growth of the re-activated bacteria from the skin care composition will positively influence the microbial flora on the skin of the subject.

The ready-to-use skin care compositions are preferably stable at room temperature for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks or more than 30 weeks. Stated differently, such compositions are preferably stable at room temperature for at least 1 month, at least 2 months, at least 3 months, at east 4 months, at least 5 months, at least 6 months or more than 6 months. As used herein, a composition is regarded as being stable if the reduction in the number of colony forming unts present in the composition after storage is less than a 3 log reduction, preferably less than a 2 log reduction, and more preferably less than a 1 log reduction. Stated differently, a composition is regarded as being stable if the reduction in the number colony forming units present in the composition after storage is less than 1000-fold, preferably less than 100-fold, and more preferably less than 10-fold relative to the number of colony forming units in the composition before storage.

In some aspects, the present invention provides a kit of parts as disclosed herein, in which the (optionally lyophilized or spray-dried) bacteria are spatially separated from the other components, e.g. the cosmetic or therapeutic components. For example, the kit of parts may be in the form of a packaging with two spatially separated chambers, wherein the first chamber contains the lyophilized or spray-dried bacteria, and the second chamber contains a composition comprising the ester of a polyethylene glycol and a fatty acid or comprising one or more compounds selected from the group consisting of an ester of a polyethylene glycol and a fatty acid, glycerol, sorbitol, lactic acid or a salt thereof, and cetearyl sulphate. Prior to use, the contents of both chambers are mixed with each other, such as for example by a consumer or patient, to provide a homogeneous skin care composition which is then applied to the skin. Alternatively, the composition comprising the live bacteria can be applied to the skin first, followed by application of the composition comprising the ester of a polyethylene glycol and a fatty acid or comprising the one or more compounds selected from the group consisting of an ester of a polyethylene glycol and a fatty acid, glycerol, sorbitol, lactic acid or a salt thereof, and cetearyl sulphate. A kit of part assembly has the advantage that the bacteria can remain in lyophilized or spray-dried form until use which is associated with a particular high storage stability of the composition. In a kit of parts assembly, it is advantageous if the weight ratio of the bacteria in the first chamber to the composition comprising the one or more compounds in the second chamber is from 1:10, 1:20, 1:30, 1:40, 1:50, 1:60, 1:70, 1:80, 1:90, or 1:100. After mixing the contents of the both chambers, a skin care composition as described herein above is created. According to the present invention a kit of parts can be provided, for example, in a Lyo-Ject® double-chamber syringe, in a V-LK® double-chamber cartridge or in a dual-chamber system as described in WO2018077598. In another preferred embodiment, the (optionally lyophilized or spray-dried) bacteria in the first chamber may be suspended in a lipid or oil. This will significantly facilitate packaging and filling. In addition, the surrounding lipid or oil will protect the bacteria from premature dehydration. Preferably, the bacteria are suspended in elthylhexyl cocoate or dicaprylyl carbonate. The weight ratio of the bacteria to the oil or lipid preferably is between 1:1 and 1:2.

In some embodiments, in any of the methods as disclosed herein, the composition comprising the ester of a polyethylene glycol and a fatty acid or comprising the one or more compounds selected from the group consisting of an ester of a polyethylene glycol and a fatty acid, glycerol, sorbitol, lactic acid or a salt thereof, and cetearyl sulphate, is applied on the skin of the subject before application of the bacteria.

It is particularly preferred that the subject is a human. In some embodiments, in particular when the skin care composition is used to improve the appearance or youthful complexion of the skin, the subject may be one in whom signs of skin aging, such as wrinkles, lines, frown lines, loss of hydration, loss of elasticity, skin sagging, blemishes, and/or pigmentation changes (“age spots”), have started to manifest. In certain embodiments, the subject may be a human subject with an age of 40 or more years, such as 45 or more years, preferably 50 or more years, such as 55 or more years, more preferably 60 or more years, such as 65 or more years, for example 70 or more years, 75 or more years, or 80 or more years. A further aspect provides a method for stimulating or boosting the growth of at least one endogenous C. acnes strain on the skin of a subject, said method comprising the topical administration of a skin care composition comprising an ester of a polyethylene glycol and a fatty acid or comprising one or more compounds selected from the group consisting of an ester of a polyethylene glycol and a fatty acid, glycerol, sorbitol, lactic acid or a salt thereof, and cetearyl sulphate to the skin of the subject. The aforementioned teachings are applicable mutatis mutandis to this aspect, in which a composition essentially as described throughout the specification, but leaving out the exogenous C. acnes, can be administered to modulate the growth of endogenous C. acnes, such as specifically certain clades or SLST types of endogenous C. acnes.

In certain embodiments, the growth of endogenous C. acnes SLST type A1, C. acnes SLST type D1, C. acnes SLST type H1 and/or endogenous C. acnes SLST type K8 can be stimulated by a skin care composition comprising an ester of a polyethylene glycol and a fatty acid, preferably a PEG stearate, even more preferably PEG40 stearate.

In certain embodiments, the growth of endogenous C. acnes SLST type H1, endogenous C. acnes SLST type K8, and/or endogenous C. acnes SLST type D1 strain can be stimulated by a skin care composition comprising glycerol.

In certain embodiments, the growth of endogenous C. acnes SLST type H1 can be stimulated by a skin care composition comprising sorbitol.

In certain embodiments, the growth of endogenous C. acnes SLST type K8 can be stimulated by a skin care composition comprising sodium lactate.

In certain embodiments, the growth of endogenous C. acnes SLST type H1 and/or endogenous C. acnes SLST can be stimulated by a skin care composition comprising sodium cetearyl sulphate.

It is apparent that there have been provided in accordance with the invention products, methods, and uses, that provide for substantial advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLE

### Example 1: Testing the Influence of Cosmetic Ingredients on C. Acnes Growth in 96-Well Plates

### Materials and Methods

### Inoculum Preparation

One day before the experiment: starter bacterial cultures of C. acnes A1, D1, H1 and K8 strains from the working cell bank (WCB) glycerol stocks were prepared in a final volume of 15 ml. The C. acnes strains were isolated from healthy volunteers and they were characterized for their growth, 16S and SLST sequence to identify the specific strain. The volumes of WCB were 1 ml for A1 and 300 μl for D1, H1, and K8. The cultures were incubated overnight at 37° C.

At the day of the experiment, after 24 h incubation, inoculums were taken out of the incubator and homogenized with vortex. 50 μl aliquots were separated from each inoculum to 1.5 ml tubes and their OD₆₀₀ was measured by Nanodrop.

Each aliquot was measured twice and its OD₆₀₀ was determined by calculating the mean value. The tubes were centrifuged (3000 rcf, 5 min) and the supernatant was discarded. The pellet was resuspended in 15 ml of PBS (phosphate buffered saline). This washing, consisting of centrifugation, discarding the supernatant, and resuspension was repeated. Then, the OD₆₀₀ was adjusted in PBS to 2 for each strain. Small aliquots were separated and read on Nanodrop again at the end of this process to ensure a starting OD₆₀₀ of 2 (+/−0.2) in the bacterial cultures.

### Media Preparation

All the ingredients were tested in either of the two different media: PBS 3% w/v Glucose and PBS 1% w/v Peptone. Additionally, all strains were grown on PBS and PBS 3% w/v Glucose 1% w/v Peptone, as negative and positive control, respectively.

TABLE 2
Media components

| Reagent | Supplier | Catalog number |
|---|---|---|
| DPBS: Dulbecco's Phosphate | Sigma Aldrich | 32129211 |
| Buffered Saline +MgCl2, CaCl2 | | |
| Glucose: α Glucose (dextrose) | Sigma Aldrich | 158968 |
| Peptone: Soy Peptone | Merck Millipore | 1.07212.5000 |

Medium was prepared at 2.5×concentration and then diluted to 1× when mixed with the ingredient and inoculum into the test plates.

PBS 7.5% w/v Glucose media was filter sterilized (Millipore pump). 50% w/v Peptone diluted in ultrapure water (Milli-Q) was prepared, autoclaved and then diluted to 2.5% w/v with PBS. PBS 7.5% w/v Glucose 2.5% w/v peptone media was prepared diluting 50% Peptone on filtered PBS 7.125% Glucose.

### Ingredient Preparation

The range of concentrations tested was based on the concentration that the chosen compound could have in the final cosmetic product. Three different concentrations were tested for each ingredient: maximum concentration, minimum concentration and a concentration in the middle between the maximum and minimum concentration (half of the highest one).

All compounds were prepared on milliQ water. 50 ml of stocks for each ingredient were prepared at double of the highest concentration to be tested. After solubilization, the ingredients were filter sterilized.

See Table 3 for all the different PEGs tested. Stock solutions of PEGs were prepared at a concentration of 8% (initial concentration). The effect of these components was evaluated in SB media (SBM). The media contains yeast extract, peptone, sugar and a mix of vitamins.

TABLE 3
Overview of the different PEG components with their corresponding
CAS number that are evaluated in the present example.

| Compound name | Cas nr |
|---|---|
| PEG 200 | 25322-68-3 |
| PEG 400 | 25322-68-3 |
| PEG 600 | 25322-68-3 |
| PEG 1500 | 25322-68-3 |
| PEG 2000 | 25322-68-3 |
| PEG 3000 | 25322-68-3 |
| PEG 6000 | 25322-68-3 |
| PEG 12000 | 25322-68-3 |
| PEG 20000 | 25322-68-3 |
| PEG 2000 MME | 9004-74-4 |
| PEG 500 DME | 24991-55-7 |
| PEG 500 MME | 9004-74-4 |
| Polyoxyl 40 stearate/PEG40 stearate | 9004-99-3 |
| PEG6000 | 25322-68-3 |
| PEG | 25322-68-3 |
| PEG-PPG-block | 9003-11-6 |

### Plate Preparation

The experiment was performed in transparent 96-well plates. Each one of them contained one media (either peptone or glucose), one strain inoculum and either of the ingredients. The final volume in each well was 200 μl: 100 μl of 2× ingredient, 80 μl of 2.5× medium and 20 μl of bacterial inoculum of OD₆₀₀. Triplicates were done for every condition. Controls without ingredient and without bacteria were included in this experiment to detect possible contaminations.

One 96-well 2 ml deep well-plate was used for each batch. The double concentrated ingredients were distributed manually on the deep well plate and the corresponding serial dilutions were performed on PBS with an automatic multichannel pipette. Then, the content of each deep well plate was distributed to 8 different 96-standard well plate with Zephyr SPE workstation (automated liquid handler).

100 μl of ingredient per well were transferred to each plate. The zephyr program contained a resuspension step on the deep well plate and a step in which the tips leaned on the sides of each well of the plates to ensure the accurate volume disposal.

Once the 100 μl of ingredient were transferred to each plate, 80 μl of either Glucose or Peptone medium was poured into the plate with Multidrop Combi Plate dispenser (Thermo Fisher). The multidrop was also used to dispense 20 μl per well of the corresponding strain inoculum, which was previously prepared as described in the previous part.

### Optical Density Measurement and Plate Incubation

After the preparation of the plates, they were sealed with transparent seal and its Optical Density was read in Spectramax microplate reader at λ=600 nm. Then, the plates were unsealed under sterility and incubated with lids for 40 h at 37 degrees Celsius.

After 40 h of incubation, the plates were shaken with a plate shaker (700 rpm, 20 minutes). Then, they were sealed and Optical Density at 600 nm read again as previously described.

### Data Analysis

The average initial OD₆₀₀ was subtracted from the average final OD₆₀₀ for each ingredient and for the controls. We then compared C. acnes growth in presence and absence of ingredient to estimate the prebiotic effect of the ingredient.

A difference of more than 0.2 between the average final OD₆₀₀ value and the average initial OD₆₀₀ value between the growth with ingredient when compared with the control of the same batch (no ingredient) was considered a remarkable positive effect of the ingredient, and therefore, it was selected as a possible prebiotic. If the growth with ingredient was 0.1 lower than the control, it was considered as an inhibitory ingredient. This criterion was followed for every concentration of every ingredient with every strain.

To interpret the values obtained from high turbidity ingredients (OD₆₀₀ above 1), ratios were obtained for each condition

$$\frac{average OD 600 t 40 h}{average OD 600 t 0 h}$$

and compared to ratio of controls. If the ratio ingredient>ratio control and standard deviation (stdv) of ingredient was not high enough to interfere with stdv of control, we considered that a positive result.

Separately, for the PEG compounds the data are presented as OD600 in function of the concentration of the compound used (g/v %) at time point 144 h. The choice was made to show the differences between the different PEG compound used at a specific timepoint and not the overall percentage of difference that could affect the final conclusion when comparing different molecules.

### Results

Effect of Glycerol on C. acnes Growth

The effect of the addition of 1% v/v glycerol was evaluated in a medium containing PBS and 1% w/v peptone as the only nitrogen source. Glycerol improved the growth of the four tested strains, i.e. A1, D1, H1 and K8, by at least a factor of four folds compared to the peptone control condition (FIG. 1).

Effect of Sorbitol on C. acnes Growth

The effect of the addition of 1% w/v sorbitol was evaluated in a medium containing PBS and 1% w/v peptone as the only nitrogen source. Sorbitol improved the growth of the four tested strains, and particularly the growth of H1 was improved by a factor of 4.5 (FIG. 2).

Effect of Sodium Lactate on C. acnes Growth

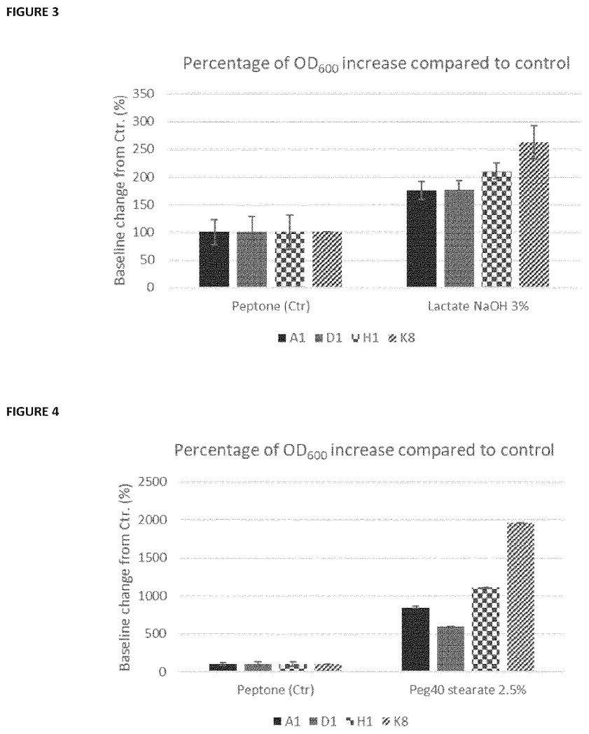

The effect of the addition of 3% w/v of sodium lactate (Lactate NaOH) was evaluated in a medium containing PBS and 1% w/v peptone as the only nitrogen source. Sodium lactate improved the growth of the four tested strains, with a higher effect on the strain K8 (FIG. 3).

Effect of PEG40 Stearate on C. acnes Growth

The effect of the addition of 2.5% w/v of PEG40 stearate was evaluated in a medium containing PBS and 1% w/v peptone as the only nitrogen source. PEG40 stearate greatly improved the growth of the four tested strains, with a higher effect on the strain K8 and H1 (FIG. 4).

Effect of Sodium Cetaryl Sulphate on C. acnes Growth

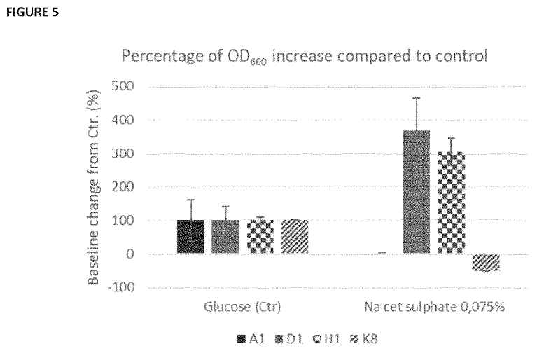

The effect of the addition of 0.075% w/v of sodium cetaryl sulphate was evaluated in a medium containing PBS and 3% w/v glucose as the only carbon source. Sodium cetaryl sulphate selectively and significantly improved the growth of D1 and H1. Negative effect was observed in the case of K8 (FIG. 5). On the other hand, none of the targeted metabolites, i.e. acetate and propionate, was detected. This could be explained by the fact that other metabolites were produced instead of the above-mentioned ones.

Effects of Different Types of PEG on C. acnes Growth

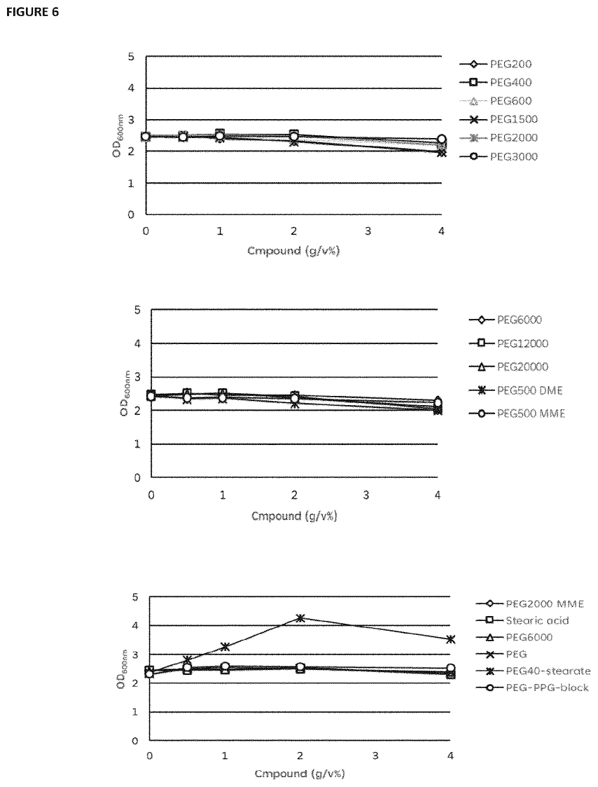

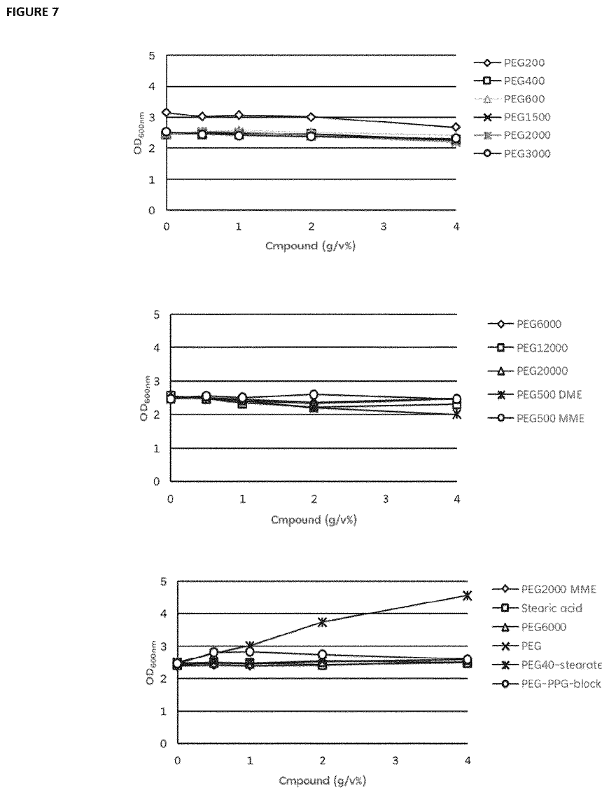

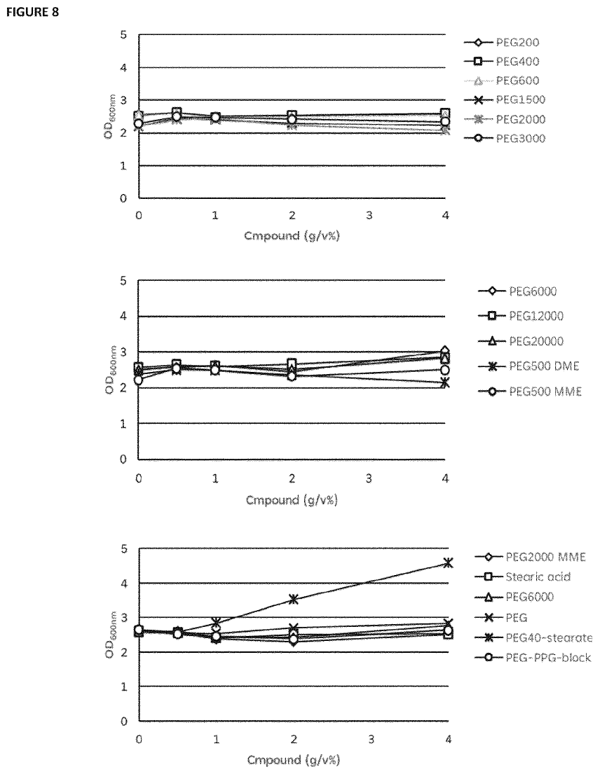

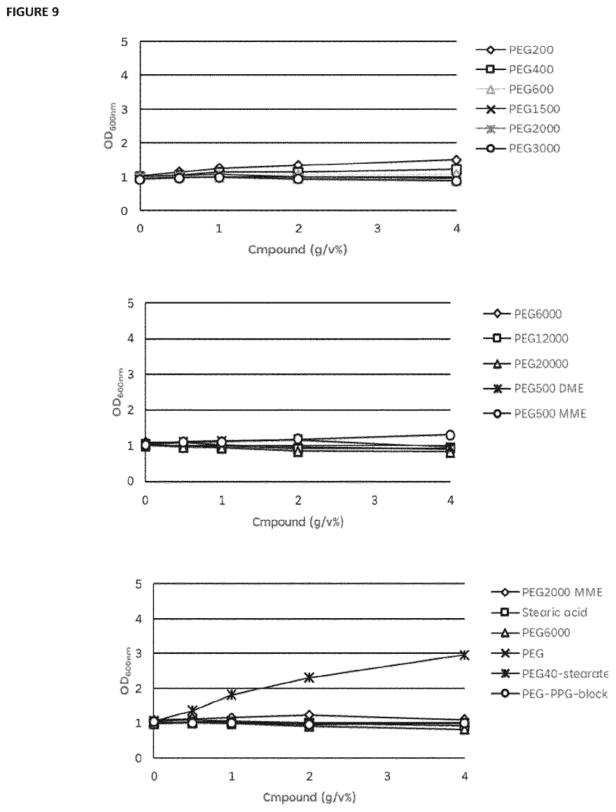

The effect of the addition of different PEG components was evaluated in the SBM which was diluted at ¼. As shown in FIGS. 6-7-8-9, only PEG40 stearate (also known as polyoxyl 40 stearate) has a dose response effect on the growth of strain A1 (FIG. 6), strain D1 (FIG. 7), strain H1 (FIG. 8) and strain K8 (FIG. 9).

### Example 2: Skin Care Compositions

The present example sets forth illustrative compositions (e.g. lotions, gels, creams, or serum) embodying the principles of the present invention, which can be suitably employed in cosmetic or pharmaceutical settings. Specific examples are provided for compositions that can be applied as day cream for acne prone skin and for an anti-ageing serum. To each of these illustrative compositions, one or more C. acnes strains are added or these strains can be provided separately to add at a later stage to the composition, so that the final concentration of the one or more C. acnes strains is at least preferably 10⁴ colony-forming units per ml (CFU/ml), preferably 10⁶-10⁹ CFU/ml, relative to the volume of the composition.

| Day cream for acne prone skin - composition 1 | wt % |
|---|---|
| Water | 76.15 |
| Sorbitol | 3 |
| Niacinamide | 5 |
| C13-16 Alcane | 7 |
| Sucrose Polystearate (and) Cetearyl Alcohol (and) | 3 |
| Olea Europaea Oil Unsaponifiables | |
| Pentylene Glycol (and) Glyceryl Caprylate/Caprate | 3 |
| Jojoba Oil | 2 |
| Allantoin | 0.5 |
| Carbomer | 0.25 |
| Sodium Phytate | 0.1 |
| Sodium Hydroxide/Citric Acid | pH Buffer |

| Day cream for acne prone skin - composition 2 | wt % |
|---|---|
| Water | 75.15 |
| PEG-40 stearate | 1 |
| Niacinamide | 5 |
| C13-16 Alcane | 7 |
| Sucrose Polystearate (and) Cetearyl Alcohol (and) | 3 |
| Olea Europaea Oil Unsaponifiables | |
| Pentylene Glycol (and) Glyceryl Caprylate/Caprate | 3 |
| Glycerin | 3 |
| Jojoba Oil | 2 |
| Allantoin | 0.5 |
| Carbomer | 0.25 |
| Sodium Phytate | 0.1 |
| Sodium Hydroxide/Citric Acid | pH Buffer |

| Day cream for acne prone skin - composition 3 | wt % |
|---|---|
| Water | 75.15 |
| Sodium Lactate | 0.3 wt % |
| Niacinamide | 5 |
| C13-16 Alcane | 7 |
| Sucrose Polystearate (and) Cetearyl Alcohol (and) | 3 |
| Olea Europaea Oil Unsaponifiables | |
| Pentylene Glycol (and) Glyceryl Caprylate/Caprate | 3 |
| Glycerin | 3 |
| Jojoba Oil | 2 |
| Allantoin | 0.5 |
| Carbomer | 0.25 |
| Sodium Phytate | 0.1 |
| Sodium Hydroxide/Citric Acid | pH Buffer |

| Day cream for acne prone skin - composition 4 | wt % |
|---|---|
| Water | 75.15 |
| Cetearyl sulphate | 0.5 wt % |
| Niacinamide | 5 |
| C13-16 Alcane | 7 |
| Sucrose Polystearate (and) Cetearyl Alcohol (and) | 3 |
| Olea Europaea Oil Unsaponifiables | |
| Pentylene Glycol (and) Glyceryl Caprylate/Caprate | 3 |
| Glycerin | 3 |
| Jojoba Oil | 2 |
| Allantoin | 0.5 |
| Carbomer | 0.25 |
| Sodium Phytate | 0.1 |
| Sodium Hydroxide/Citric Acid | pH Buffer |

| Anti-ageing serum - composition 5 | wt % |
|---|---|
| Distilled Water | 89.3 |
| Sorbitol | 5 |
| Pentylene Glycol (and) Glyceryl Caprylate/Caprate | 3 |
| Niacinamide | 2 |
| Xanthan Gum | 0.3 |
| Sodium Hyaluronate (HMW) | 0.2 |
| Hydrolyzed Hyaluronic Acid (LMW) | 0.1 |
| Sodium Phytate | 0.1 |
| Sodium Hydroxide/Citric Acid | pH Buffer |

| Anti-ageing serum - composition 6 | wt % |
|---|---|
| Distilled Water | 88.3 |
| PEG 40 stearate | 1 |
| Glycerin | 5 |
| Pentylene Glycol (and) Glyceryl Caprylate/Caprate | 3 |
| Niacinamide | 2 |
| Xanthan Gum | 0.3 |
| Sodium Hyaluronate (HMW) | 0.2 |
| Hydrolyzed Hyaluronic Acid (LMW) | 0.1 |
| Sodium Phytate | 0.1 |
| Sodium Hydroxide/Citric Acid | pH Buffer |

| Anti-ageing serum - composition 7 | wt % |
|---|---|
| Distilled Water | 89.0 |
| Sodium lactate | 0.3 |
| Glycerin | 5 |
| Pentylene Glycol (and) Glyceryl Caprylate/Caprate | 3 |
| Niacinamide | 2 |
| Xanthan Gum | 0.3 |
| Sodium Hyaluronate (HMW) | 0.2 |
| Hydrolyzed Hyaluronic Acid (LMW) | 0.1 |
| Sodium Phytate | 0.1 |
| Sodium Hydroxide/Citric Acid | pH Buffer |

| Anti-ageing serum - composition 8 | wt % |
|---|---|
| Distilled Water | 88.8 |
| Cetearyl sulfate | 0.5 |
| Glycerin | 5 |
| Pentylene Glycol (and) Glyceryl Caprylate/Caprate | 3 |
| Niacinamide | 2 |
| Xanthan Gum | 0.3 |
| Sodium Hyaluronate (HMW) | 0.2 |
| Hydrolyzed Hyaluronic Acid (LMW) | 0.1 |
| Sodium Phytate | 0.1 |
| Sodium Hydroxide/Citric Acid | pH Buffer |

### REFERENCES

Fitz-Gibbon et al. 2013. Propionibactium acnes strain populations in the human skin microbiome associated with acne. J Invest Dermatol 133 (9).
Holmberg et al. 2009. Biofilm formation by Propionibacterium acnes is a characteristic of invasive isolates. Clin Microbiol Infect 15:787-95.
Johnson and Cummins, 1972. Cell wall composition and deoxyribonucleic acid similarities among the anaerobic coryneforms, classical proprionibacteria and strains of Arachnia propionica. J Bacteriol. 109(3): 1047-66.
Lodes et al., 2006. Variable expression of immunoreactive surface proteins of Propionibacterium acnes. Microbiology 152:3667-3681.
Lomholt and Kilian, 2010. Population and genetic analysis of Propionibacterium acnes identifies a subpopulation and epidemic clones associated with acne. Plos One 5(8) e12277.
Mc Dowell at al., 2005. Propionibacterium acnes types I and II represent phylogenetically distinct groups. J Clin Microbiol 43(1):326-334.
McDowell et al., 2008. A new phylogenetic group of Propionibacterium acnes. J Med Microbiol 57:218-224.
McDowell et al., 2012. An expanded multilocus sequence typing scheme for Propionibacteium acnes: investigation of ‘pathogenic’, ‘commensal’ and antibiotic resistant strains. Plos One 7 (7) e41480.
Scholz et al., 2014. A novel high-resolution single locus sequence typing scheme for mixed populations of Propionibacterium acnes in vivo. Plos One, 9 (8) e104199.
Valanne at al. 2005. CAMP factor homomologues in Propionibacterium acnes: a new protein family differentially expressed by types I and II. Microbiology 151:1369-1379.

1. A skin care composition comprising:
a) live bacteria of at least one Cutibacterium acnes (C. acnes) strain; and
b) an ester of a polyethylene glycol and a fatty acid.

2. The skin care composition of claim 1, wherein the bacteria are lyophilized or spray-dried.

3. The skin care composition of claim 1, wherein the fatty acid is a saturated fatty acid and/or the fatty acid is a C16-C18 fatty acid.

4.-5. (canceled)

6. The skin care composition of claim 1, wherein the fatty acid is a stearic acid.

7. The skin care composition of claim 1, wherein the ester of a polyethylene glycol and a fatty acid is PEG40 stearate.

8. The skin care composition of claim 1, wherein the composition comprises:
between 0.01 wt % and 10 wt %, preferably between 0.06 wt % and 5 wt %, of the ester of a polyethylene glycol and a fatty acid relative to the total weight of the skin care composition.

9. (canceled)

10. The skin care composition of claim 1, wherein the at least one C. acnes strain is selected from C. acnes SLST type A1 strain, C. acnes SLST type D1 strain, C. acnes SLST type H1 strain, or C. acnes SLST type K8 strain.

11. The skin care composition of claim 1, comprising:
a) live bacteria of C. acnes SLST type A1 strain, C. acnes SLST type D1 strain, C. acnes SLST type H1 strain, C. acnes SLST type K8 strain, or a mixture thereof; and
b) an ester of a polyethylene glycol and a fatty acid, preferably a PEG stearate, even more preferably PEG40 stearate.

12. The skin care composition of claim 1, comprising at least 10⁴ colony-forming units per ml (CFU/ml) of each live C. acnes bacterial strain, preferably about 10⁴-10⁹ CFU/ml of each live C. acnes bacterial strain, relative to the volume of the skin care composition.

13. The skin care composition of claim 1, wherein the skin care composition is in the form of a gel, cream, ointment or lotion.

14. A method of treating and/or preventing a condition comprising administering the skin care composition of claim 1 to a subject, wherein the condition is selected from the group consisting of acnes, oily skin, progressive macular hypomelanosis, dandruff, atopic eczema, atopic dermatitis, and rosacea.

15. A method of treating and/or preventing an oxidative stress-associated skin disease, comprising administering the skin care composition of claim 1 to a subject, optionally wherein the oxidative stress-associated skin disease is selected from the group comprising actinic keratosis (AK), basal cell carcinoma (BCC), squamous cell carcinoma (SCC), dandruff, seborrheic dermatitis, acne, inflammation, dermatitis, psoriasis, eczema, rosacea, urticaria, and vitiligo.

16. A method for improving the appearance of the skin of a subject and/or for modulating the sebum production of skin cells of a subject and/or for maintaining a healthy or youthful appearance of the skin in a subject, said method comprising topical administration of the skin care composition of claim 1 to an area of the subject's skin.

17. The method of claim 14, wherein the ester of the polyethylene glycol and the fatty acid are applied on the skin of the subject before application of the bacteria.

18. The method of claim 14, wherein the subject is a human subject.

19. A method for stimulating or boosting the growth of at least one endogenous C. acnes strain on the skin of a subject, said method comprising topical administration of:
a skin care composition comprising an ester of a polyethylene glycol and a fatty acid
to the skin of the subject, optionally wherein the skin care composition further comprises live bacteria of at least one Cutibacterium acnes (C. acnes) strain.

20. A method for stimulating or boosting the growth of at least one C. acnes bacterial strain in vitro, said method comprising administration of a composition comprising a polyethylene glycol ester of a fatty acid to said at least one C. acnes bacterial strain in vitro.

21. The method of claim 20 wherein the fatty acid is a saturated fatty acid and/or wherein the fatty acid is a C16-C18 fatty acid.

22.-23. (canceled)

24. The method of claim 20, wherein the fatty acid is a stearic acid.

25. The method of claim 20, wherein the ester of a polyethylene glycol and a fatty acid is PEG40 stearate.


SEQUENCE LISTING (8 sequences)

SEQ ID NO: 1 - DNA, 21 bp, synthetic construct
  misc_feature 1..21: note = Forward primer
  source 1..21: mol_type = other DNA
cagcggcgctgctaagaactt

SEQ ID NO: 2 - DNA, 20 bp, synthetic construct
  misc_feature 1..20: note = Reverse primer
  source 1..20: mol_type = other DNA
ccggctggcaaatgaggcat

SEQ ID NO: 3 - DNA, 54 bp, synthetic construct
  misc_feature 1..54: note = SLST-Adapter-Forward primer
  source 1..54: mol_type = other DNA
tcgtcggcagcgtcagatgtgtataagagacagcagcggcgctgctaagaactt

SEQ ID NO: 4 - DNA, 54 bp, synthetic construct
  misc_feature 1..54: note = SLST-Adapter-Reverse primer
  source 1..54: mol_type = other DNA
gtctcgtgggctcggagatgtgtataagagacagccggctggcaaatgaggcat

SEQ ID NO: 5 - DNA, 484 bp, Propionibacterium acnes
  source 1..484: mol_type = genomic DNA
gttgcacaccagggggtcaacttggcgtcctcagttcaaaattgattcaaactaacagttccatgtcgggaaacagcaccaggaagctcgtgacatatcgtctttcattgcgagaaacatcttacttatgtacatttctaagctatagcgtctacccttgtcagacccaggacgatgggtgtcacatctcctttctagtcaacctaagagaggaggaaatgccgcgatatatgttccaccctgtcatcacgaaggccaccacaatctatcccagaacagccggcacttcactcacgatgccccgatgctggattcctattgtcgcccttattagggcaagcggtgccagtagcagaatatgtcacctcaacaactcgatccacccctgcccattacatgggtaacatatccatggaggttcgatgtatactcgaggatacagtcgtccatcacgcccgcctacatacccattacatcagcatag

SEQ ID NO: 6 - DNA, 484 bp, Propionibacterium acnes
  source 1..484: mol_type = genomic DNA
gttgcacaccagggggtcaacttggcgtcctcagttcaaaattgattcaaactaacagttccatgtcgggaaacagcaccaggaaactcgtgacatatcgtctttcattgcgagaaacatcttacttatgtacatttctaagctatagcgtctacccttgtcagacccaggacgatgggtgtcacatcccctttctagtcaacctaagagaggaggaaatgccgcgatatatgttccgccctgtcatcacgaagaccaccacaatctatcccagaacagccggcacttcactcacgatgccccgatgctggattcctattgtcgcccttattagggcaagcggtgccagtagcagaatatgtcacctcaacaactcgatccacccctgcccattacatgggtaacatatccatggaggttcgatgtatacttgaggatacagtcgtccatcacgcccacctacatacccattacatcagcatag

SEQ ID NO: 7 - DNA, 484 bp, Propionibacterium acnes
  source 1..484: mol_type = genomic DNA
gttgcacaccagggggtcaacttggcgtcctcagttcaaaattgattcaaactaacagttccatgtcgggaaacagcaccaggaagctcgtgacatatcgtctttcattgcgagaaacatcttacttatgtacatttctaagctatatcgtctacccttgtcagacccaggacgatggatgtcacatcccctttctagtcaacctaagagaggaggaaatgccgcgatatatgttccaccctgtcatcacgaaggccaccacaatctatcccagaacagccggcacttcactcacgatgccccgatgctggattcctattgtcgcccttattagggcaagcggtgccagtagcagaatatgtcacctcaacaactcgatccacccctgcccattacatgggtaacatatccatggaggttcgatgtatattcgaggatacagtcgtccatcacgcccgcctacatacccattacatcagcatag

SEQ ID NO: 8 - DNA, 484 bp, Propionibacterium acnes
  source 1..484: mol_type = genomic DNA
attgcacaccagggggtcaacttggtgtcctcagttcaaaattggttcaaactaacggttccgtgtcgggaaacagcaccagaaaactcgtgacatatcgtctttcattgcgagaaacatcttacttatacacatttctaagctatattgtctacccctgtcagacccaggacgatgggtgtcatatcccctttccagtcaacctaagaagggaggaaatgccgcgatatatgttccgccctgtcatcatgaatgccaccacaatctatcccggaacagccgtacttcacccaccatgccccgatgctggattcctattgtcgcccttattagagcaagcggtgccagcagcagaatatttcacctcagcaactcgatccgctcctgcccattacatgggtaacatatccatggaggtacgatgtatgcatcgaggatgcagtcgtctactatgcccgcctacatacccattccatcagcatag